(19) United States
(12) Patent Application Publication
(10) Pub. No.: US 2026/0060801 A1
(43) Pub. Date: Mar. 5, 2026

(54) PROSTHETIC VALVES AND DELIVERY ASSEMBLIES WITH POSITIONING AND STABILIZATION MEMBERS

(71) Applicant: EDWARDS LIFESCIENCES CORPORATION, Irvine, CA (US)

(72) Inventors: David Maimon, Atlit (IL); Eitan Atias, Tel Aviv (IL); Ofir Witzman, Harish (IL); Noa Axelrod Manela, Kadima (IL); Alon Ben-Yosef, Ramot Menashe (IL); Tomer Saar, Pardes Hanna-Karkur (IL)

(73) Assignee: EDWARDS LIFESCIENCES CORPORATION, Irvine, CA (US)

(21) Appl. No.: 19/378,819

(22) Filed: Nov. 4, 2025

Related U.S. Application Data
(63) Continuation of application No. PCT/US2024/027571, filed on May 2, 2024.
(60) Provisional application No. 63/464,147, filed on May 4, 2023.

(51) Int. Cl. A61F 2/24

(52) U.S. Cl. CPC A61F 2/2418; A61F 2/2433; A61F 2210/0014; A61F 2230/0004; A61F 2250/0098

(57) ABSTRACT

The present disclosure relates to devices designed to aid in accurate positioning and deployment of prosthetic valves inside a native annulus. In an example, a delivery assembly comprises a prosthetic valve comprising a frame movable between radially compressed and expanded configurations, and a delivery apparatus. The delivery apparatus includes an outer delivery shaft, and at least one elongated positioning member extending through, and axially movable relative to, the outer delivery shaft. The elongated positioning member is configured to position a distal end thereof radially outward to the prosthetic valve and axially distal to an outflow end of the frame. In some examples, the delivery apparatus can further include at least one sensor attached to the elongated positioning member.

### CROSS-REFERENCE TO RELATED APPLICATIONS

This application is a continuation of International Application No. PCT/US2024/027571, filed May 2, 2024, which claims the benefit of U.S. Provisional Application No. 63/464,147, filed May 4, 2023, which is incorporated by reference herein.

### FIELD

The present disclosure relates to prosthetic valves and delivery assemblies equipped with positioning and/or stabilization members, and to methods of utilization thereof.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches, such as transcatheter aortic valve replacement (TAVR), are used in various procedures to deliver prosthetic medical devices to locations inside the body that are not readily accessible by surgery or where access without surgery is desirable.

Transcatheter aortic valve replacement (TAVR) is one example of a minimally-invasive surgical procedure used to replace a native aortic valve. In one specific example of the procedure, an expandable prosthetic heart valve is mounted in a crimped state on the distal end of a delivery apparatus and advanced through the patient's vasculature (e.g., through a femoral artery and the aorta) to the heart. The prosthetic heart valve is positioned within the native valve and expanded to its functional size.

### SUMMARY

Numerous TAVR techniques are known in the art, including techniques which are percutaneous, trans-arterial, trans-venous, trans-cardiac, trans-atrial, trans-ventricular, and/or trans-apical. A key factor in such transcatheter valve deployment is properly positioning the prosthetic device, e.g., accurately positioning a prosthetic valve within the native heart valve annulus. For example, a prosthetic valve which is implanted too deep relative to the native annulus may cause conduction disturbances. In another example, a prosthetic valve is misaligned within the native annulus, it may dislodge from the site of implantation and/or result in undesirable paravalvular leakage and regurgitation along spaced formed between the valve and the surrounding tissue. Thus, it is desirable to provide devices and methods by which a prosthetic valve can be accurately positioned relative to the native annulus during the implantation procedure.

In one of its basic configurations, a delivery assembly comprises a prosthetic valve and a delivery apparatus comprising at least one elongated positioning member. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the delivery apparatus can comprise a handle.

In some examples, the delivery apparatus can comprise an outer delivery shaft optionally extending distally from the handle.

In some examples, the at least one elongated positioning member optionally extends through, and is optionally axially movable relative to, the outer delivery shaft.

In some examples, the delivery apparatus can comprise at least one sensor attached to the at least one elongated positioning member.

In some examples, the at least one elongated positioning member is optionally configured to position a distal end thereof radially outward to the prosthetic valve and axially distal to an outflow end of the frame.

In one of its basic methods, a method comprises advancing a delivery assembly that comprises a delivery apparatus carrying a prosthetic valve in a radially compressed configuration, to a native heart valve. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises extending at least one elongated positioning member of the delivery apparatus through and distally to an outer delivery shaft of the delivery apparatus, until a distal end of the at least one elongated positioning member interacts with a proximal abutment surface of the native heart valve.

In some examples, the method comprises acquiring measurement signals from at least one sensor attached to the at least one elongated positioning member.

In some examples, the method optionally comprises expanding the prosthetic valve within an annulus of the native heart valve, while the distal end of the at least one elongated positioning member is radially spaced away from the prosthetic valve.

In one of its basic configurations, a delivery assembly comprises a prosthetic valve and a delivery apparatus comprising a positioning balloon. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the delivery apparatus can comprise a handle.

In some examples, the delivery apparatus can comprise an outer delivery shaft extending distally from the handle.

In some examples, the positioning balloon is optionally movable between a deflated state and an inflated state.

In some examples, the delivery apparatus can comprise an inflation tube coupled to and in fluid communication with the positioning balloon.

In some examples, the inflation tube optionally extends through, and is optionally axially movable relative to, the outer delivery shaft.

In some examples, the inflation tube is optionally configured to position the positioning balloon radially outward to the prosthetic valve, such that at least a portion of the positioning balloon extends axially distally to an outflow end of the frame.

In one of its basic configurations, a delivery assembly comprises a prosthetic valve and a delivery apparatus comprising a stabilization filter. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the delivery apparatus can comprise a handle.

In some examples, the delivery apparatus can comprise an outer delivery shaft optionally extending distally from the handle and a stabilization filter.

In some examples, the stabilization filter is optionally configured to transition between collapsed state and a deployed state.

In some examples, the stabilization filter is optionally positioned proximal to the prosthetic valve.

In some examples, the stabilization filter can comprise a plurality of pores.

In one of its basic configurations, a delivery assembly comprises a delivery apparatus and a prosthetic valve comprising one or more positioning struts. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the one or more positioning struts can be optionally configured to transition between a compacted state and a deployed state.

In some examples, the frame can extend between an inflow end and an outflow end.

In some examples, the delivery apparatus can comprise a handle.

In some examples, the delivery apparatus can comprise an inner capsule and an outer capsule.

In some examples, the inner capsule is optionally configured to retain the prosthetic valve in the radially compressed configuration therein.

In some examples, the outer capsule optionally has an inner diameter greater than an outer diameter of the inner capsule.

In some examples, the one or more positioning struts are optionally configured to extend radially away from the frame in the deployed state.

In some examples, the prosthetic valve, the inner capsule, and the outer capsule, are axially movable relative to each other.

In one of its basic configurations, a delivery assembly comprises a prosthetic valve and a delivery apparatus comprising one or more positioning arms extending through a nosecone shaft lumen of a nosecone shaft of the delivery apparatus. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the frame optionally extends between an inflow end and an outflow end.

In some examples, the delivery apparatus can comprise a handle.

In some examples, the nosecone shaft optionally extends distally from the handle.

In some examples, the delivery apparatus can comprise a nosecone attached to a distal portion of the nosecone shaft.

In some examples, the nosecone shaft can comprise one or more side opening formed at the distal portion of the nosecone shaft.

In some examples, the one or more positioning arms are optionally configured to transition between a compacted state and a deployed state.

In some examples, the one or more positioning arms are optionally axially movable relative to the nosecone shaft and the nosecone.

In some examples, the one or more positioning arms are optionally configured to assume the compacted state when fully retained inside the nosecone shaft lumen.

In some examples, the one or more positioning arms are optionally configured to assume a deployed state when at least a portion thereof extends through the one or more side openings.

In some examples, the one or more positioning arms are optionally configured to extend radially away from the nosecone shaft in the deployed state.

In one of its basic configurations, a delivery assembly comprising a prosthetic valve and a delivery apparatus comprising one or more positioning arms attached to a nosecone of the delivery apparatus. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the prosthetic valve can comprise a frame movable between a radially compressed and a radially expanded configuration.

In some examples, the frame optionally extends between an inflow end and an outflow end.

In some examples, the delivery apparatus can comprise a handle.

In some examples, the delivery apparatus can comprise a nosecone shaft extending distally from the handle.

In some examples, the nosecone is optionally attached at a nosecone proximal end thereof to a distal portion of the nosecone shaft.

In some examples, the one or more positioning arms are optionally attached to the nosecone at fixed ends thereof, and can optionally extend proximally therefrom to free ends of the one or more positioning arms.

In some examples, the delivery apparatus can comprise one or more tensioning members attached to the free ends of the one or more positioning arms, and optionally extending proximally therefrom.

In some examples, the nosecone shaft optionally defines a nosecone shaft lumen.

In some examples, the nosecone shaft can comprise one or more side opening formed at the distal portion of the nosecone shaft.

In some examples, the one or more positioning arms are optionally configured to transition between a compacted state and a deployed state.

In some examples, the one or more positioning arms are optionally configured to assume the compacted state when the one or more tensioning members attached thereto are tensioned.

In some examples, the one or more positioning arms are optionally configured to assume the deployed state when tension is released from the one or more tensioning members.

In some examples, the one or more positioning arms are optionally configured to extend radially away from the nosecone and the nosecone shaft in the deployed state.

In one of its basic configurations, a delivery assembly comprises a delivery apparatus and a prosthetic valve comprising an outer skirt which comprises a circumferential mesh. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the delivery apparatus can comprise a capsule.

In some examples, the prosthetic valve can comprise a frame movable between a radially compressed and a radially expanded configuration.

In some examples, the outer skirt is optionally disposed around the frame.

In some examples, the circumferential mesh is optionally configured to transition between a compacted state and an expanded free state.

In some examples, the circumferential mesh is optionally configured to assume the compacted state when the prosthetic valve is retained inside the capsule, and to assume the expanded free state when the prosthetic valve is deployed out of the capsule.

In one of its basic configurations, a prosthetic valve comprises a frame comprising a plurality of struts that comprise a plurality of inflow vertical struts. This basic configuration can preferably be provided with any one or more of the features described elsewhere herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the frame is optionally movable between a radially compressed and a radially expanded configuration.

In some examples, the frame optionally extends between an inflow end and an outflow end.

In some examples, the frame can comprise a plurality of intersecting struts.

In some examples, the plurality of struts can comprise a plurality of angled struts and a plurality of vertical struts.

In some examples, the plurality of vertical struts can comprise the plurality of inflow vertical struts.

In some examples, the inflow vertical struts are optionally defined between cells of the frame extending from the inflow end.

In some examples, the plurality of vertical struts can comprise a plurality of outflow vertical struts.

In some examples, the outflow vertical struts are optionally defined between cells of the frame extending from the outflow end.

In some examples, the inflow vertical struts optionally define an inflow strut length which is greater than an outflow struts length defined by the outflow vertical struts.

In one of its basic methods, a method comprises advancing a delivery assembly that comprises a delivery apparatus carrying a prosthetic valve in a radially compressed configuration, to a native heart valve. This basic method can preferably be provided with any one or more of the steps described elsewhere herein, in particular with those of the examples described hereafter. However, it should be understood that the basic method can preferably also be provided with any one or more of the steps shown in the figures and/or described in conjunction with the figures, either in addition to or alternatively to the steps of the examples described hereafter.

In some examples, the method comprises contacting an abutment surface of the native heart valve by at least one positioning member of the delivery apparatus.

In some examples, the method comprises identifying axial position of an annulus of the native heart valve by monitoring the at least one positioning member under fluoroscopy.

In some examples, the method comprises positioning an inflow end of the prosthetic valve at an axial position relative to the at least one positioning member while monitoring both a frame of the prosthetic valve and the at least one positioning member under fluoroscopy.

In some examples, the method optionally comprises expanding the prosthetic valve within the annulus.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of a frame of the prosthetic valve of FIG. 1A.

FIG. 2 shows an exemplary delivery assembly comprising a delivery apparatus carrying a prosthetic valve.

FIG. 3 shows an exemplary delivery assembly comprising a positioning member.

FIG. 4 shows an exemplary delivery assembly comprising a distally-oriented sensor attached to a positioning member.

FIG. 5 shows an exemplary delivery assembly comprising a laterally-oriented sensor attached to a positioning member.

FIG. 6 shows an exemplary delivery assembly comprising axially-spaced sensors attached to a positioning member.

FIG. 7 shows an exemplary delivery assembly comprising a first sensor attached to a positioning member and a second sensor attached to another component of the delivery assembly.

FIG. 8 shows an exemplary delivery assembly comprising an inflatable positioning balloon.

FIG. 9 shows an exemplary delivery assembly comprising a stabilization filter.

FIG. 10 shows an exemplary delivery assembly comprising a prosthetic valve equipped with positioning struts.

FIG. 11 shows a distal portion of a delivery apparatus comprising an inner capsule disposed inside an outer capsule.

FIG. 12A shows a portion of a prosthetic valve disposed inside the inner and outer capsules, with the positioning arms retained in a compacted state.

FIG. 12B shows the prosthetic valve of FIG. 12A, with the positioning arms in a free state.

FIG. 13A shows an exemplary delivery assembly with positioning arms retained in a compacted state inside a nosecone shaft.

FIG. 13B shows the delivery assembly of FIG. 13A, with the positioning arms extending radially outward through side openings of the nosecone shaft.

FIG. 14A shows an exemplary delivery assembly with positioning arms extending proximally from the nosecone, retained in a compacted state by tensioned tensioning members.

FIG. 14B shows the delivery assembly of FIG. 14A, with the positioning arms extending radially outward relative to the nosecone, while the tensioning members are released.

FIG. 15 shows an exemplary delivery assembly comprising an outer skirt equipped with a circumferential mesh,

FIG. 16 shows a frame of an exemplary prosthetic valve, having inflow vertical struts which are longer than the outflow vertical struts.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (e.g., the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

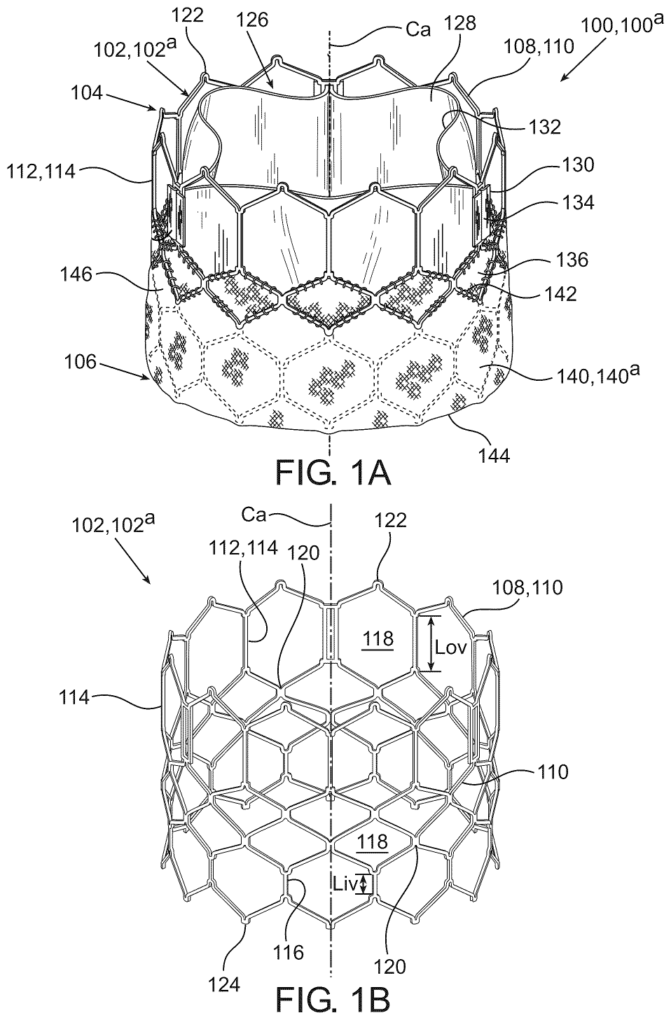

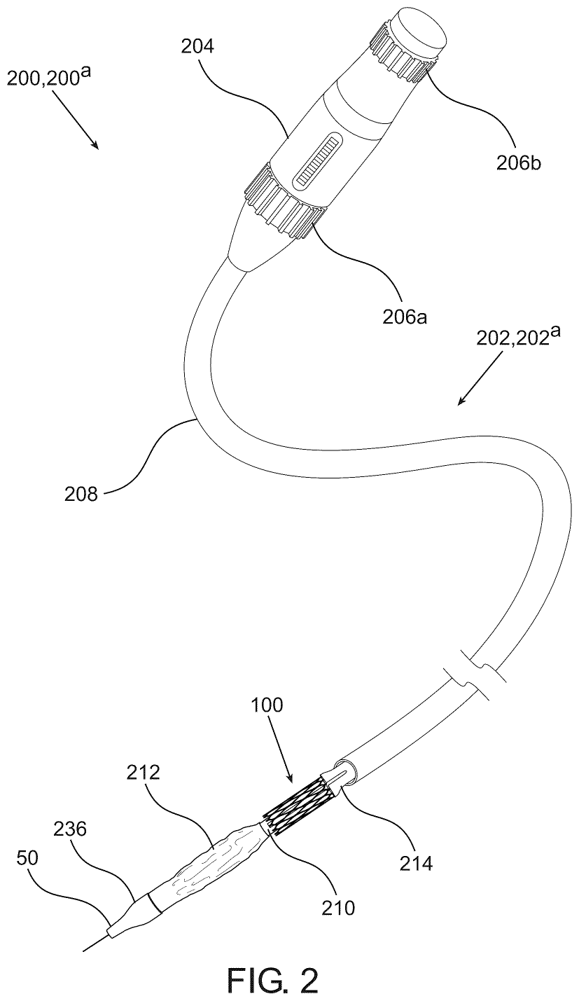

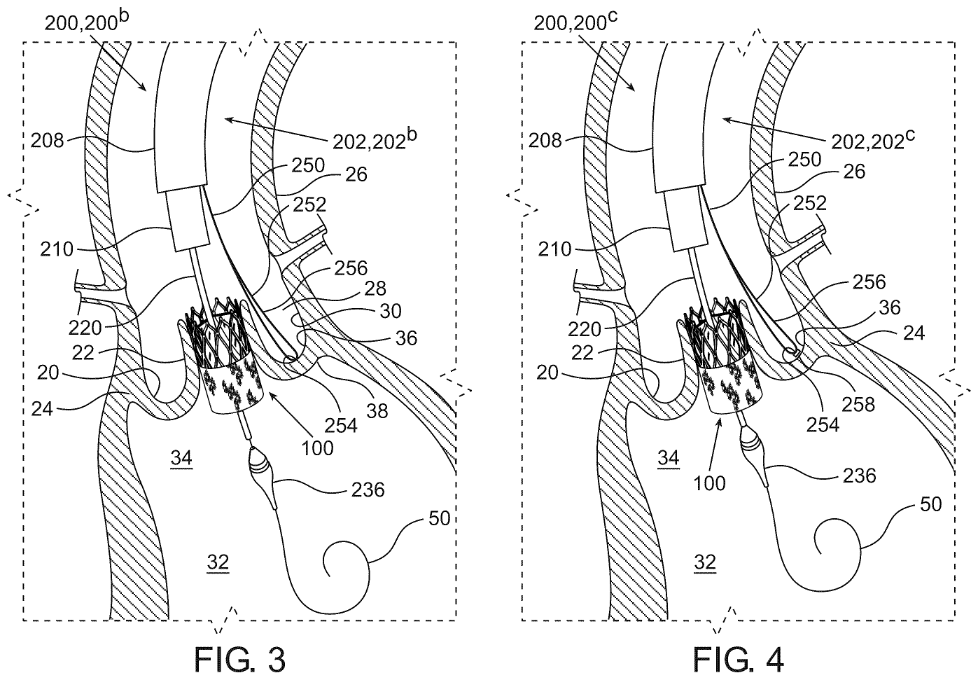

FIGS. 1A and 1B show perspective views of a prosthetic valve 100 with and without soft components attached thereto, according to some examples. FIG. 2 shows a perspective view of a delivery assembly 200, according to some examples. The delivery assembly 200 can include the prosthetic valve 100 and a delivery apparatus 202. The prosthetic valve 100 can optionally be on or releasably coupled to the delivery apparatus 202. The delivery apparatus can optionally include a handle 204 at a proximal end thereof, and a nosecone shaft 220 (concealed from view in FIG. 2, but exposed, for example, in FIG. 3) extending distally from the handle 204, having a nosecone 236 attached to its distal end.

The term “prosthetic valve”, as used herein, refers to any type of a prosthetic valve deliverable to a patient's target site over a catheter, which is radially expandable and compressible between a radially compressed, or crimped, state, and a radially expanded state. Thus, a prosthetic valve 100 can be crimped or retained by a delivery apparatus 202 in a compressed state during delivery, and then expanded to the expanded state once the prosthetic valve 100 reaches the implantation site. The expanded state may optionally include a range of diameters to which the valve may expand, between the compressed state and a maximal diameter reached at a fully expanded state. Thus, a plurality of partially expanded states may relate to any expansion diameter between radially compressed or crimped state, and maximally expanded state.

A prosthetic valve 100 of the current disclosure may include any prosthetic valve configured to be mounted within the native aortic valve, the native mitral valve, the native pulmonary valve, and the native tricuspid valve. While a delivery assembly 200 described in the current disclosure, includes a delivery apparatus 202 and a balloon expandable prosthetic device, such as prosthetic valve 100, it should be understood that the delivery apparatus 202 according to any example of the current disclosure can optionally be used for implantation of other prosthetic devices aside from prosthetic valves, such as stents or grafts.

A catheter deliverable prosthetic valve 100 can optionally be delivered to the site of implantation via the delivery assembly carrying the valve 100 in a radially compressed or crimped state, toward the target site, to be mounted against the native anatomy, by expanding the prosthetic valve 100 via various expansion mechanisms. Balloon expandable valves generally involve a procedure of inflating a balloon within a prosthetic valve, thereby expanding the prosthetic valve 100 within the desired implantation site. Once the valve is sufficiently expanded, the balloon is deflated and retrieved along with the delivery apparatus 202. Self-expandable valves include a frame that is shape-set to automatically expand as soon an outer retaining capsule, which may be also defined as the distal portion of an outer shaft or the distal portion of a delivery shaft, is withdrawn proximally relative to the prosthetic valve.

Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of the delivery apparatus, controlled via a handle for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

The delivery assembly can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

FIGS. 1A-1B show an example of a prosthetic valve 100, which can optionally be a balloon expandable valve or any other type of valve, illustrated in an expanded state. The prosthetic valve 100 can comprise an outflow end 104, an inflow end 106, and a central longitudinal axis Ca extending in a direction from the inflow end 106 to the outflow end 104. In some instances, the outflow end 104 is the proximal end of the prosthetic valve 100, and the inflow end 106 is the distal end of the prosthetic valve 100. Alternatively, depending for example on the delivery approach of the valve, the outflow end can be the distal end of the prosthetic valve, and the inflow end can be the distal end of the proximal valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The prosthetic valve 100 comprises an annular frame 102 movable between a radially compressed configuration and a radially expanded configuration, and a leaflet assembly 126 mounted within the frame 102. The frame 102 can be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a plastically-deformable materials, the frame 102 can be crimped to a radially compressed state on a balloon catheter 210, and then expanded inside a patient by an inflatable expansion balloon 212 or equivalent expansion mechanism. Alternatively or additionally, the frame 102 can be made of shape-memory materials such as, but not limited to, nickel-titanium alloy (e.g., Nitinol). When constructed of a shape-memory material, the frame 102 can be crimped to a radially compressed state and restrained in the compressed state by insertion into a shaft or equivalent mechanism of a delivery apparatus 202.

In the example illustrated in FIGS. 1A-1B, the frame 102 is an annular, stent-like structure comprising a plurality of intersecting struts 108. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 108 may be any elongated member or portion of the frame 102. The frame 102 can include a plurality of strut rungs that can collectively define one or more rows of cells 118. The frame 102 can have a cylindrical or substantially cylindrical shape having a constant diameter from the inflow end 106 to the outflow end 104 as shown, or the frame can vary in diameter along the height of the frame, as disclosed in U.S. Pat. No. 9,155,619, which is incorporated herein by reference.

The end portions of the struts 108 are forming apices 122 at the outflow end 104 and apices 124 at the inflow end 106. The struts 108 can intersect at additional junctions 120 formed between the outflow apices 122 and the inflow apices 124. The junctions 120 can be equally or unequally spaced apart from each other, and/or from the apices 122, 124, between the outflow end 104 and the inflow end 106.

At least some of the struts 108 can be pivotable or bendable relative to each other, so as to permit frame expansion or compression. For example, the frame 102 can be formed from a single piece of material, such as a metal tube, via various processes such as, but not limited to, laser cutting, electroforming, and/or physical vapor deposition, while retaining the ability to collapse/expand radially in the absence of hinges and like.

A leaflet assembly 126 of the prosthetic valve 100 can optionally include a plurality of prosthetic leaflets 128 (e.g., three leaflets), positioned at least partially within the frame 102, and configured to regulate flow of blood through the prosthetic valve 100 from the inflow end 106 to the outflow end 104. While three leaflets 128 arranged to collapse in a tricuspid arrangement, are shown in the example illustrated in FIG. 1A, it will be clear that a prosthetic valve 100 can include any other number of leaflets 128.

The inflow or cusp edges of the leaflets 128 can optionally be secured to the frame 102 directly or indirectly, such as by being sutured directly to the frame, being sutured to an inner skirt, and/or via one or more connecting skirts. Further examples and methods of attaching skirts and seal members to a frame, as well as method and techniques for coupling leaflets 128 to the frame 102, with or without connecting skirts, are disclosed in US Pat. Publication No. 2018/0028310, which is incorporated herein by reference.

Adjacent leaflets 128 can optionally be arranged together to form commissures 134 that are coupled (directly or indirectly) to respective portions of the frame 102, thereby securing an upper portion (e.g., above the scalloped line) of the leaflet assembly 126 to the frame 102. In some examples, each leaflet 128 can optionally comprise opposing tabs 130. Each tab 130 can optionally be secured to an adjacent tab 130 of an adjacent leaflet 128 to form a commissure 134 that is secured to the frame 102. The tabs 130 can be folded in various manners, for example to form radially extending layers and circumferentially extending layers facing the frame. Radially extending layers can extends radially inward from a location on the frame 102 to free edge 132, also termed coaptation edges, of the leaflet.

During valve cycling, the leaflets 128 can articulate at the inner most edges of the tab layers, which helps space the leaflets away from the frame 102 during normal operation of the prosthetic valve. This is particular advantageous in cases where the prosthetic valve 100 is not fully expanded to its nominal size when implanted in a patient. As such, the prosthetic valve 100 can be implanted in a wider range of patient annulus sizes. Further details regarding transcatheter prosthetic valves, including the manner in which leaflets 128 can be coupled to the frame 102 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

According to some examples, the prosthetic valve 100 can optionally further comprise at least one skirt or sealing member. An inner skirt 136 can be secured to the inner surface of the frame 102, configured to function, for example, as a sealing member to prevent or decrease perivalvular leakage. An inner skirt 136 can further function as an anchoring region for the leaflets 128 to the frame 102, and/or function to protect the leaflets 128 against damage which may be caused by contact with the frame 102, for example during valve crimping or during working cycles of the prosthetic valve 100. Additionally, or alternatively, the prosthetic valve 100 can optionally comprise an outer skirt 140 mounted on the outer surface of the frame 102, configure to function, for example, as a sealing member retained between the frame 102 and the surrounding tissue of the native annulus against which the prosthetic valve is mounted, thereby reducing risk of paravalvular leakage (PVL) past the prosthetic valve 100.

The outer skirt 140 can optionally comprise a base layer 146 extending from an outer skirt inflow end 144 to an outer skirt outflow end 142. Any of the inner skirt 136 and/or base layer 146 of the outer skirt 140 can be made of various suitable biocompatible materials, such as, but not limited to, various synthetic materials (e.g., PET) or natural tissue (e.g. pericardial tissue). In some cases, the inner skirt 136 can optionally be formed of a single sheet of material that extends continuously around the inner surface of frame 102. In some cases, the optionally outer skirt 140 or a base layer 146 thereof can be formed of a single sheet of material that extends continuously around the outer surface of frame 102.

Struts 108 comprise angled struts 110, and optionally vertical struts 112. The term “vertical strut” refers to a strut that generally extends in an axial direction parallel to central longitudinal axis Ca, while the term “angled strut” generally refers to a strut that can extend at an angle relative to an axial line intersecting therewith along a plane defined by the frame 102. It is to be understood that the term “angled strut” encompasses both linear angled struts and curved struts.

FIG. 1B shows an example of a frame 102 that includes at least two types of vertical struts 112, namely outflow vertical struts 114 defined between cells 118 extending from the outflow end 104, and inflow vertical struts 116 defined between cells 118 extending from the inflow end 106. As illustrated, the cells 118 extending from the inflow end 106 and from the outflow end 104 can optionally define, in some examples, hexagonal opening therein, while the frame can optionally further include at least one additional row of diamond-shaped cells therebetween.

Various exemplary examples for prosthetic valves 100, delivery assemblies 200, and/or components thereof, can be referred to, throughout the specification, with superscripts, for ease of explanation of features that refer to such exemplary examples. It is to be understood, however, that any reference to structural or functional features of any assembly, device or component, without a superscript, refers to these features being commonly shared by all specific exemplary examples that can be also indicated by superscripts. In contrast, features emphasized with respect to an exemplary implementation of any assembly, device or component, including prosthetic valve 100 and/or delivery assembly 200, referred to with a superscript, may be optionally shared by some but not necessarily all other exemplary examples. For example, prosthetic valve 100^{a} is an exemplary implementation of prosthetic valve 100, and thus includes all of the features described for prosthetic valve 100 throughout the current disclosure, except that while a prosthetic valve 100 can be generally provided with any type of struts 108, including angled struts and/or vertical struts 112 of any shape and size, prosthetic valve 100^{a} includes outflow vertical struts 114 having an outflow vertical strut length Lov and inflow vertical struts 116 having an inflow vertical strut length Liv, such that the length Lov of the outflow vertical struts 114 is greater than the length Liv of the inflow vertical struts 116 (i.e., Lov>Liv).

While examples of a delivery assembly described in the current disclosure, are shown to include an exemplary delivery apparatus and a balloon expandable prosthetic valve, it should be understood that a delivery apparatus according to any example of the current disclosure can be used for implantation of other prosthetic devices aside from prosthetic valves, such as stents or grafts.

A delivery assembly comprising any delivery apparatus described throughout the current disclosure can be utilized, for example, to deliver a prosthetic aortic valve for mounting against the native aortic annulus or against a prosthetic valve previously implanted in a native aortic valve, to deliver a prosthetic mitral valve for mounting against the native mitral annulus or against a prosthetic valve previously implanted in a native mitral valve, or to deliver a prosthetic valve for mounting against any other native annulus or against a prosthetic valve previously implanted in any other native valve.

FIG. 2 illustrates a delivery assembly 200 that a delivery apparatus 202 adapted to deliver a prosthetic valve, such as prosthetic valve 100 described above with respect to FIGS. 1A-1B. According to some examples, the delivery apparatus 202 includes a handle 204 and at least one catheter extending therefrom, configured to carry a prosthetic valve 100 in a crimped state through the patient's vasculature. An exemplary delivery assembly 200^{a} optionally comprises an exemplary delivery apparatus 202^{a} configured to carry a balloon expandable prosthetic valve. The delivery apparatus 202^{a} can optionally comprise a balloon catheter 210 having an inflatable expansion balloon 212 mounted on its distal end. A balloon expandable prosthetic valve 100 can optionally be carried in a crimped state over the balloon catheter 210.

In some examples, a delivery apparatus 202 further comprises an outer delivery shaft 208. Optionally, an outer delivery shaft 208 of a delivery apparatus 202^{a} can concentrically extend over the balloon catheter 210. In some examples, delivery apparatus 202^{a} can optionally further comprise a push shaft 214 disposed over the balloon catheter 210, optionally between the balloon catheter 210 and the outer delivery shaft 208.

The outer delivery shaft 208, the push shaft 214, and the balloon catheter 210, can optionally be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic valve 100 from the outer delivery shaft 208.

A delivery apparatus 202 can optionally further include a nosecone 236 carried by a nosecone shaft 220 (hidden from view in FIG. 2, but shown in any of FIGS. 3-10 for example). In the case of delivery apparatus 202^{a}, the nosecone shaft 220 can optionally extend through a lumen of the balloon catheter 210.

The proximal ends of the balloon catheter 210, the outer delivery shaft 208, the push shaft 214, and/or the nosecone shaft 220, can optionally be coupled to the handle 204. During delivery of the prosthetic valve 100, the handle 204 can be maneuvered by an operator (e.g., a clinician or a surgeon) to axially advance or retract components of the delivery apparatus 202, such as the nosecone shaft 220, the outer delivery shaft 208, and for delivery apparatus 202^{a} also the balloon catheter 210 and/or the push shaft 214, through the patient's vasculature and/or along the target site of implantation, as well as to inflate the expansion balloon 212 mounted on the balloon catheter 210, so as to expand the prosthetic valve 100, and to deflate the balloon 212 and retract the delivery apparatus 202 once the prosthetic valve 100 is mounted in the implantation site.

The handle 204 can optionally include a steering mechanism configured to adjust the curvature of the distal end portion of the delivery apparatus 202. In the illustrated example, the handle 204 includes an adjustment member, such as the illustrated rotatable knob 206^{a}, which in turn is operatively coupled to the proximal end portion of a pull wire (not shown). The pull wire can extend distally from the handle 204 through the outer delivery shaft 208 and has a distal end portion affixed to the outer delivery shaft 208 at or near the distal end of the outer delivery shaft 208. Rotating the knob 206^{a} can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 214 relative to the balloon catheter 210 in the case of delivery apparatus 202^{a}. The handle can include additional knobs to control additional components of the delivery apparatus 202, such as positioning members that will be described in greater detail below.

The prosthetic valve 100 can be carried by the delivery apparatus 202 during delivery in a crimped state, and expanded, for example by balloon inflation, to secure it in a native heart valve annulus (such as an aortic annulus 24 shown in FIG. 3) or against a previously implanted prosthetic valve (for example, during valve-in-valve implantation procedures). In an exemplary implantation procedure utilizing delivery assembly 200^{a}, the prosthetic valve 100 is initially crimped over the balloon catheter 210, proximal to the expansion balloon 212. Because prosthetic valve 100 is crimped at a location different from the location of balloon 212, prosthetic valve 100 can be crimped to a lower profile than would be possible if it was crimped on top of balloon 212. This lower profile permits the clinician to more easily navigate the delivery assembly 200^{a} (including crimped prosthetic valve 100) through a patient's vasculature to the treatment location. The lower profile of the crimped prosthetic valve is particularly helpful when navigating through portions of the patient's vasculature which are particularly narrow, such as the iliac artery.

The expansion balloon 212 can optionally be secured to balloon catheter 210 at the balloon's proximal end, and to either the balloon catheter 210, the nosecone shaft 220, or the nosecone 236 at its distal end. The distal end portion of the push shaft 214 is positioned proximal to the outflow end 104 of prosthetic valve 100.

When reaching the site of implantation, the deflated balloon 212, carrying crimped valve 100 thereover, can be advanced to the target site to expand the prosthetic valve. Prior to balloon 212 inflation, the push shaft 214 is optionally advanced distally, allowing its distal end portion to contact and push against the outflow end 104 of prosthetic valve 100, pushing the valve 100 distally therewith. The distal end of push shaft 214 is dimensioned to engage with the outflow end 104 of prosthetic valve 100 in a crimped configuration of the valve. In some examples, the distal end portion of the push shaft 214 can optionally be flared radially outward, to terminate at a wider-diameter that can contact the prosthetic valve 100 in its crimped state. Optionally, push shaft 214 can then be advanced distally, pushing the prosthetic valve 100 therewith, until the crimped prosthetic valve 100 is disposed around the balloon 212, at which point the balloon 212 can be inflated to radially expand the prosthetic valve 100. Once the prosthetic valve 100 is expanded to its functional diameter within a native annulus or within a previously implanted prosthetic valve, the balloon 212 can be deflated, and the delivery apparatus 202 can be retrieved from the patient's body.

In particular examples, any exemplary delivery assembly of the current disclosure can optionally be packaged in a sterile package that can be supplied to end users for storage and eventual use. In particular examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

As mentioned above, while specific methods described and illustrated herein address replacement of an aortic valve, any of the exemplary delivery assemblies 200 described throughout the current disclosure can be used to aid in the accurate positioning and deployment of implants relative to all cardiac valves, as well as relative to other orifices and body lumens, such as the orifices of all the major arteries and veins related to the heart (including but not limited to the superior and inferior vena cava, pulmonary arteries and veins, coronary sinus, innominate artery, common carotid arteries, and subclavian arteries).

FIG. 3 shows an exemplary delivery assembly 200^{b} utilized for deployment of a prosthetic valve 100 within a native annulus 24. Delivery assembly 200 can optionally be advanced into a patient via the femoral artery (not shown) and then through the aorta 26 and the aortic root 28 toward the aortic valve 20. In some examples, the distal portion of delivery assembly 200 can optionally be further advanced through the annulus 24 and native leaflets 22 of the aortic valve 20 and into the left ventricle 32, such as into the left ventricle outflow tract (LVOT) 34 of the left ventricle 32, as depicted in FIG. 3.

As described above, proper positioning the prosthetic valve 100 can advantageously ensure successful implantation of the valve within the native heart valve annulus. Examples of delivery assemblies 200 disclosed herein can include delivery apparatuses 202 and/or prosthetic valves 100 equipped with positioning and/or stabilization components that can be utilized during deployment of the prosthetic valve and implantation within a native heart valve.

Delivery assembly 200^{b} is an exemplary implementation of delivery assembly 200, and thus includes all of the features described for delivery assembly 200 throughout the current disclosure, except that the delivery apparatus 202^{b} of delivery assembly 200^{b} further comprises at least one elongated positioning member 250 extending distally from the handle 204. The elongated positioning member 250 can extend through a shaft or a catheter of the delivery apparatus 202^{b}, such as through a lumen of an outer delivery shaft 208.

In some examples, delivery assembly 200^{b} can optionally be configured for delivery of a balloon expandable valve, and delivery apparatus 202^{b} can be implemented according to any of the examples described above for delivery apparatus 202^{a}. In some examples, the elongated positioning member 250 can optionally extend through a space formed between the balloon catheter 210 and the outer delivery shaft 208 as illustrated in FIG. 3.

The positioning member 250 can optionally be independently maneuvered through a sheath or catheter of the delivery apparatus 200^{b}, such as the outer delivery shaft 208 or any other shaft, and extend out of the shaft (e.g., out of outer delivery shaft 208), for example, via operation of the handle 204 by the clinician. While positioning member 250 is illustrated to extend out of outer delivery shaft 208, in some examples, the delivery apparatus can optionally include multiple shafts which can be disposed adjacent to each other within the same sheath. Thus, it is to be understood that any reference to any type of a positioning member extendable through outer delivery shaft 208 as disclosed herein, can similarly refer to the positioning member extending through any other sheath of shaft of the delivery apparatus.

A positioning member 250 can optionally be axially movable relative to other components of the delivery assembly 200, such as the prosthetic valve 100, the outer delivery shaft 208, and/or balloon catheter 210. The handle 204 can optionally include a mechanism (not shown) to control axial movement of the elongated positioning member 250. While a single elongated positioning member 250 is illustrated in FIG. 3, it is to be understood that any other number is contemplated, including any plurality of two or more elongated positioning members 250 circumferentially spaced from each other around the prosthetic valve 100.

The native valve 20 can define a proximal abutment surface 36, defined as a proximally facing surface of the native valve 20, and can be formed by the cusps or a proximally-facing portion of the native leaflets 22 extending from the annulus 24. An elongated positioning member 250 can optionally be extended from the distal end of outer delivery shaft 208, to interact with an abutment surface at the region of implantation, in order to guide placement and positioning of the prosthetic valve 100 within the host valve (e.g., native aortic valve). For example, an elongated positioning member 250 can optionally be advanced to contact and rest on the proximal abutment surface 36 of the native heart valve 20, to assist in accurately positioning the prosthetic valve 100. The elongated positioning member 250 can optionally be axially movable distal to the outer delivery shaft 208, and can optionally be advanced such that its distal portion is spaced radially away from the outer delivery shaft 208 and/or the prosthetic valve 100.

In some examples, a distal end 254 of the elongated positioning member 250 is constructed to be atraumatic (e.g., blunt or otherwise lacking sharp edges) to avoid damage to the surrounding anatomy during operation. For example, the elongated positioning member 250 can optionally have a contoured distal end 254 having a circular shape, oval shape (e.g., spoon-shaped), elliptical shape, C-shape, J-shape, or any other arcuate shape, such that an area of contact between the distal end 254 and an abutment surface 36 (e.g., cusp portion) can be increased. In some examples, the elongated positioning member 250 can optionally be formed from a plurality of wires, for example, as a pair of wires that are bent laterally to converge to contact or attach to each other at distal end 254. In some examples, the elongated positioning member comprises a catheter or sheath with an atraumatic distal end (example not illustrated).

In some examples, the elongated positioning member 250 comprises a loop 252 at a distal end portion thereof. The loop 252 can optionally define slanted side segments 256 and a bottom curved segment 254 therebetween. Advancement of the elongated positioning member 250 can optionally be performed such that the bottom curved segment 254 is pressed against the proximal abutment surface 36. In some examples, the elongated positioning member 250 can optionally be a pre-shaped wire or cable, for example, a wire formed of a shape memory material, such as Nitinol. Thus, as the elongated positioning member 250 as advanced out of the outer delivery shaft 208, an end portion thereof, such as loop 252, can adopt their predetermined shape. In some examples, elongated positioning member 250 can optionally be formed of other materials, such as a metal (e.g., steel, titanium, etc.), metal alloy (e.g., cobalt chromium alloy, etc.), plastic, or any combination thereof.

Delivery assembly 200 can optionally be advanced towards the site of implantation, such as a native aortic valve 20, from the ascending aorta 26. The elongated positioning member 250 can optionally be retained within the outer delivery shaft 208 prior to reaching the site of implantation. Upon reaching the aortic valve 20, the (one or more) elongated positioning member 250 can optionally be advanced from the distal end of the outer delivery shaft 208 toward, but optionally spaced from, the proximal abutment surface 36 (e.g., the cusp floor of the native leaflet 22). Simultaneous with the advancement of the elongated positioning member 250 or sequential thereto (e.g., before or after), the prosthetic valve 100 can optionally be advanced from the distal end of the outer delivery shaft 208, for example along with balloon catheter 210, toward and optionally through the native aortic annulus 24, as shown in FIG. 3. The advancement of elongated positioning member 250 can optionally continue until the elongated positioning member 250 reaches the proximal abutment surface 36.

Using tactile feedback from the elongated positioning member 250 created by the contact of its distal end, such as the bottom curved segment 254, with the proximal abutment surface 36, the clinician can confirm the position of the native annulus, relative to which the prosthetic valve 100 can be adequately positioned. Conventional techniques for prosthetic valve positioning inside the native annulus include injection of contrast media into the region of implantation to visualized anatomical structures which are otherwise invisible under fluoroscopy. Utilization of exemplary positioning members disclosed herein can provide visual indication of the annular level, relative to which the prosthetic valve 100 can be maneuvered and positioned, without the need to introduce contrast media into the blood stream.

Exemplary positioning members disclosed herein are visible under X-ray, such that once contact with the native annulus is identified, the prosthetic valve 100 can be axially moved relative to the position of the corresponding positioning member, indicative of a desired position of the prosthetic valve 100 relative to the native annulus 24. For example, tactile feedback from the elongated positioning member 250, as described above, can provide an indication of contact with the proximal abutment surface 36. Since the elongate positioning member 250 can optionally be formed of a metallic wire or other radiopaque material, the position of the bottom curved segment 254, visible under fluoroscopy, may be indicative of the position of the native annulus 24, such as its proximal abutment surface 36. The frame 102 of a prosthetic valve 100, which also comprises metallic material visible under fluoroscopy, can be then advanced to position the inflow end 106 at a desired position, relative to the annulus 24, by tracking, in real-time, the positions of the frame 102 and the bottom curved segment 252 of loop 252 under an adequate imaging modality, such as fluoroscopy, without the need to inject a contrast agent, such as Barium or other type of contrast agent, into the patient blood stream during the procedures, which can advantageously increase the safety of the procedure and reduce costs of materials.

When a positioning arm, according to any examples disclosed herein, is configured to contact a specific surface or region of the annulus 24, additional anatomical characteristics can be accounted for during positioning of the prosthetic valve. For example, if a positioning member, such as elongate positioning member 250, is configured to contact a proximal abutment surface 36, the thickness of the native annulus 24 can be accounted for if positioning of the valve 100 is desired relative to a distal abutment surface 38 or relative to a center of the native annulus 24.

When the prosthetic valve 100 is positioned at a desired position, relative to the imaged portion of positioning member 250, corresponding to the native annulus 24, the clinician can optionally inflate an expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100 into the desired position within the native annulus 24. During prosthetic valve expansion, the elongated positioning member 250 can optionally remain engaged with (e.g., pressed against) the proximal abutment surface 36, optionally absorbing at least some of the force exerted by the delivery assembly 200 on aortic valve 20 during the implantation procedure.

In some examples, prior to full expansion of the prosthetic valve 100, the elongated positioning member 250 can optionally be retracted from the aortic root 28, thereby allowing the prosthetic valve 100 to fully expand against the inner walls 30 of the aortic root 28. The elongated positioning member 250 can be optionally (but not necessarily) retracted back into the outer delivery shaft 208, the balloon 212 (if used) can be deflated to a reduced diameter, and the catheter delivery apparatus 202ⁱ can be then withdrawn from the patient, leaving the prosthetic valve 100 implanted within the native valve 20.

It is to be understood that the clinician can rely on additional positioning techniques, such as fluoroscopy, echocardiography, etc. For example, during initial advancement of the delivery assembly 200 into the heart, the clinician can use fluoroscopic, echocardiographic, and/or other imaging methods to provide visual confirmation of the orientation and position of the delivery assembly 200, including components thereof such as prosthetic valve 100, and/or positioning member 250, relative to the annulus 24 or other anatomical region of interest. The clinician can also use the fluoroscopic, echocardiographic, and/or other imaging methods to provide visual confirmation of the orientation and position of various components of the delivery assembly in addition to the tactile feedback provided by the positioning member, e.g., during the positioning of the valve 100 described herein using the positioning member or other positioning or stabilization components and devices. The tactile feedback thus provides the clinician with another important sensory cue to the relative position of the positioning member/prosthetic valve with respect to the annulus.

In some examples, the elongated positioning member 250 can be relatively conspicuous under applicable image-guidance modalities, such as fluoroscopic, echocardiographic, and/or other imaging methods. As mentioned above, the elongated positioning member 250 can optionally be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the elongated positioning member 250 is either made of a radiopaque material or comprises radiopaque markers. This can assist, in some cases, in identification of contact of the elongated positioning member 250 with the abutment surface 36. For example, when using fluoroscopy or other appropriate imaging technique, the clinician can see the distal portion of the elongated positioning member 250, such as loop 252 and/or bottom curved segment 254 thereof, begin to buckle, bend or otherwise deform in shape, when engaged with (i.e., pressed against) the proximal abutment surface 36. Thus, contact of the elongated positioning member 250 can be identified by tactile feedback and/or visually from fluoroscopy or other image guidance.

FIG. 4 shows an exemplary delivery assembly 200^{c}. Delivery assembly 200^{c} is an exemplary implementation of delivery assembly 200, and can be similar to any example described above with respect to delivery assembly 200^{b} that includes at least one elongated positioning member 250, except that the delivery apparatus 202^{c} further comprises at least one sensor 258 attached to the elongated positioning member 250, the sensor oriented distally (i.e., facing a surface that can be substantially orthogonal to the longitudinal axis defined by the elongated positioning member), configured to contact the proximal abutment surface 36 when the elongated positioning member 250 is pressed against the surface 36. As mentioned above with respect to delivery assembly 200^{b}, more than one elongated positioning member 250 can be similarly included in delivery apparatus 202^{c}. When delivery apparatus 202^{c} includes a plurality of elongated positioning members 250, one, some, or all of the positioning members 250 can optionally include sensor(s) 258 attached thereto.

In some examples, the sensor 258 is a force sensor, configured to provide feedback regarding the force applied by the elongated positioning member 250 on the surrounding anatomy. The sensor 258 can optionally be attached to a distal end of the elongated positioning member 250. In some examples, when the elongated positioning member 250 comprises a loop 252, the force sensor 258 is attached to the bottom curved segment 254, configured to provide feedback regarding the force applied by the loop 252 of the positioning member 250 on the proximal abutment surface 36.

In some examples, the delivery apparatus 202^{c} can include one or more optional communication devices, a sensor data unit (not shown), and one or more user output devices (not shown). The term “communication device”, as used herein, means any device that allows communication therethrough, passively and/or actively. In some examples, this includes wires, optical fibers, or wireless communication terminals. A communication device can be configured to allow electrical communication (e.g., via a conductive material, such as a wire) and/or optical communication (e.g., via an optical fiber).

A communication device can optionally be implemented as an insulated wire extending from the sensor 258, such as along and optionally attached to the length of the elongated positioning member. For simplicity, such examples of communication devices are not illustrated to extend from sensors 258 throughout the illustrations. In some examples, the elongated positioning member 250 can optionally also serve as a communication device. For example, the elongated positioning member 250 can optionally be formed of a conductive wire that can be insulated along its length, but exposed to the sensor 258 at the sensor's 258 attachment point.

In some examples, the sensor 258 is in communication with a sensor data unit. In some examples, the sensor 258 is in wired communication with a sensor data unit via a communication device. In some examples, the sensor 258 is in wireless communication with a sensor data unit. In some examples, the sensor 258 is operated by the sensor data unit such that the sensing of sensor 258 is performed in cooperation with the sensor data unit. For example, in an implementation where the sensor 258 comprises a strain gauge bridge, the sensor data unit applies a predetermined excitation voltage at the input leads of the bridge and measures the voltage at the output leads of the bridge. The sensor data unit then determines the applied force, or pressure, from the measured output voltage. In some examples, the sensor 258 comprises dedicated circuitry for operation and the sensor data unit receives the measured data from the sensor 258. In some examples, the sensor 258 is in wireless communication with an external computing device.

In some examples, the one or more user output devices comprise respective visual and/or auditory informative elements configured to generate a visual and/or auditory information, such as, such as a display, LED lights, speakers and the like. These options are not limiting, and other feedback can also be provided to a user or operator of delivery apparatus 202^{c} by the one or more user output devices.

In some examples, the sensor data unit is in communication with an external system (not shown). Such an external system can include a processor and a memory. The memory has stored therein a plurality of instructions, which when run by the processor causes the processor to perform a plurality of predetermined functions. In some examples, communication between a sensor data unit and an external system is performed via dedicated antennas and/or connection to various networks.

In some examples, a sensor data unit can optionally include one or more processors and a memory, the memory having a plurality of instructions stored therein. When the one or more processors reads the plurality of instructions, the plurality of instructions cause the one or more processors to perform the functions of the sensor data unit. In some examples, the sensor data unit is implemented on a microcontroller, with one or more peripherals of the microcontroller in communication with the at least one sensor 258.

In some examples, the at least one sensor 258 comprises at least one force sensor. The term “force sensor”, as used herein, means any sensor that senses the magnitude of a force, or pressure, applied thereto. It is particularly noted that anywhere in the disclosure (in the description and/or claims) where the term “force sensor” is used, this can optionally include a pressure sensor. The force sensor 258 can optionally comprise, in some examples: a piezoresistive sensor, such as a strain gauge or a strain gauge bridge, the resistance of the piezoresistive sensor being a respective predetermined function of the force applied thereto; a piezoelectric sensor, the voltage at the output of the piezoelectric sensor being a respective predetermined function of the force applied thereto; a capacitive sensor, the capacitance of the capacitive sensor being a respective predetermined function of the force applied thereto; and/or an optical sensor, the optical interferometry of the optical sensor being a respective predetermined function of the force applied thereto.

As described above, in one example the measurements of sensor(s) 258 are performed in cooperation with a sensor data unit. Alternatively, the measurements of sensor(s) 258 are performed by dedicated circuitry of sensor(s) 258 and transmitted to a sensor data unit.

When contact with the proximal abutment surface 36 is identified by the force measurement of force sensor 258, a visual or auditory indication thereof can be the one or more user output devices, which can optionally include, as mentioned, LED lights or other indications that may be implemented on the handle 204. Based on such indication, further advancement of the elongated positioning member 250 can optionally be halted, after which positioning and implantation of the prosthetic valve 100 can proceed as described above.

Prosthetic valve 100 expansion against the surrounding tissue may pose a variety of risks associated with a mismatch between the valve's expansion diameter and the surrounding tissue. One complication is related to valve over-expansion, which may exert excessive radial forces on the surrounding anatomy, resulting in potential damage to the tissue or even annular rupture. On the other hand, valve under-expansion might increase the risk of aortic valve or mitral valve regurgitation. Inappropriate expansion may also result in unfavorable hemodynamic performance across the valve 100, such as increased pressure gradients or flow disturbances resulting from diameter mismatch, which may be associated with increased risk of thrombus formations.

Thus, in order to avoid the deleterious effects of either annular rupture, inferior hemodynamic performance or valve regurgitation, arising due to either over-expansion or under-expansion, respectively, of the prosthetic valve 100, a clinician should be able to control the degree of valve expansion according to real-time feedback received during the procedure, indicating, for example, current forces exerted by the valve on its surroundings, or reactive forces of the surrounding tissue, resisting valve expansion.

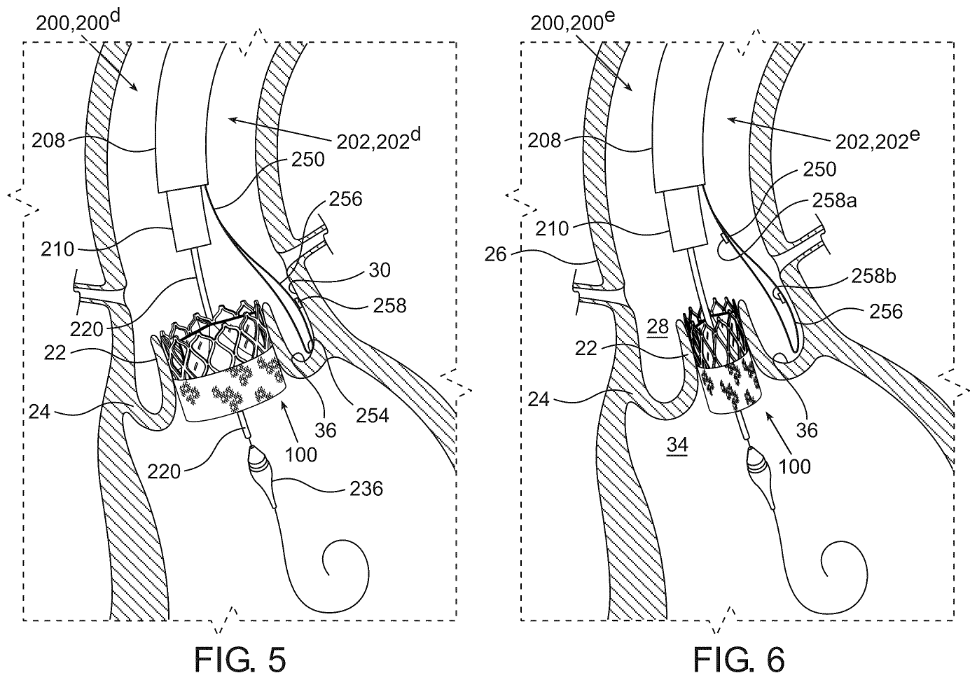

FIG. 5 shows an exemplary delivery assembly 200^{d}. Delivery assembly 200^{d} is an exemplary implementation of delivery assembly 200, and can be similar to any example described above with respect to delivery assembly 200^{c} that includes at least one elongated positioning member 250 having at least one force sensor 258 attached thereto, except that the force sensor 258 of delivery apparatus 202^{d} is oriented laterally (e.g., facing a surface that can be substantially parallel to the longitudinal axis defined by the elongated positioning member), configured to contact an axially extending anatomical surface between the prosthetic valve and the anatomy against which it is expanded.

It is to be understood that other than the position and orientation of the force sensor 258 on elongated positioning member 250, the delivery assembly 200^{d} can optionally be implemented structurally and functionally according to any example described above for delivery assembly 200^{c}.

In some examples, the force sensor 258 can optionally be attached to a portion of the elongated positioning member 250 which is exposed out of the outer delivery shaft 208, and is distal to the outflow end 104 of prosthetic valve 100 during the valve's expansion procedure, yet may be proximal to the proximal abutment surface 36. In some examples, the force sensor 258 is attached to a side segment 256 of loop 252. In some examples, the force sensor 258 is configured to face radially away from the central longitudinal axis Ca of the prosthetic valve 100 during the valve expansion procedure, such as towards the aortic root inner wall 30 as illustrated in FIG. 5. In some examples, the force sensor 258 is configured to face radially towards the central longitudinal axis Ca of the prosthetic valve 100 during the valve expansion procedure (example not illustrated), such as towards a native leaflet 22 positioned between the prosthetic valve 100 and the elongated positioning member 250, and/or a component of the prosthetic valve 100 itself, such as outer skirt 140 and/or frame 102.

When elongated positioning member 250 is engaged with the proximal abutment surface 36, for example, as shown in FIG. 5, the force sensor 258 may be positioned between the prosthetic valve 100 and the aortic root inner wall 30. When the valve 100 is initially compressed, it is sufficiently spaced from the aortic root inner wall 30 such that the force sensor 258 is not yet forcibly pressed against any side of the anatomy and/or the prosthetic valve 100. As the prosthetic valve 100 is expanded, for example due to balloon inflation, or any other expansion mechanism (such as release from a dedicated capsule for self-expandable valves, or mechanical actuation in the case of mechanically-expandable valves), the frame 102 pushes against the loop 252 or other distal portion of the positioning member 250 closer to the aortic root inner wall 30, until the sensor 258 is forcibly pressed therebetween, which will cause the measured force reading of the sensor 258 to increase.

The sensor data unit can optionally output a signal indicative of the force or pressure exerted by the valve 100 on the surrounding tissue. Based on the determined magnitude of force applied by the valve 100 during such expansion on its surroundings, the clinician can determine the maximal expansion diameter and optionally halt further expansion so as not to exceed a maximal predetermined threshold, above which damage may be inflicted to the surrounding tissues.

While a distally oriented force sensor 258 is illustrated in FIG. 4 and described above with respect to delivery assembly 200^{c}, and a laterally oriented force sensor 258 is illustrated in FIG. 5 and described above with respect to delivery assembly 200^{d}, it is to be understood that more than one sensor 258 can optionally be coupled to different regions of the same elongated positioning member 250. For example, an elongated positioning member 250 can optionally be equipped both with a distally-oriented force sensor 258, optionally coupled to a bottom curved segment 254 of a loop 252, and a laterally-oriented force sensor 258, optionally coupled to a side segment 256 of the loop 252, which can allow utilization of the sensors 258 of the elongated positioning member 250 both for identification of contact with the proximal abutment surface 36, and measurement of the force exerted by the prosthetic valve 100 on the surrounding anatomy during expansion thereof, as described above. Moreover, when a delivery apparatus 202 includes a plurality of elongated positioning members 250, each can optionally be equipped with a different type of force-sensor, such that at least one of the elongated positioning members 250 can optionally be equipped with a distally-oriented force sensor 258, and at least another one of the elongated positioning members 250 can optionally be equipped with a laterally-oriented force sensor 258.

Inappropriate valve expansion may also result in unfavorable hemodynamic performance across the valve 100, such as increased pressure gradients. Thus, it may be desirable to further provide the clinician with real-time trans-valvular pressure gradients measurements during the valve implantation procedure.

FIG. 6 shows an exemplary delivery assembly 200^{e}. Delivery assembly 200^{e} is an exemplary implementation of delivery assembly 200, and can be similar to any example described above with respect to delivery assembly 200^{d} that includes at least one elongated positioning member 250 having at least one sensor 258 attached thereto, except that instead of a single laterally-oriented sensor 258 is illustrated for delivery apparatus 202^{d}, attached to a region of the positioning member 250 configured to be positioned distally to the outflow end 104 of the valve 100 during valve expansion procedure, the elongated positioning member 250 of delivery assembly 200^{e} is shown to include at least two pressure sensors 258a and 258b, attached to different regions of the positioning member 250 axially spaced from each other.

Unlike the sensor 258 of delivery apparatus 202^{d}, the pressure sensors 258 of delivery apparatus 202^{e} are attached to regions of positioning member 250 which are exposed out of the outer delivery shaft 208, yet configured to remain proximal to the outflow end 104 of prosthetic valve 100 during the valve's expansion procedure. The pressure sensors 258 are configured to measure blood pressure along regions proximal to the prosthetic valve 100. Pressure gradient downstream prosthetic valve 100 can be thus measured by the difference between pressure readings of both sensors 258b and 258a. In some examples, unlike the sensor 258 of some exemplary examples of delivery apparatus 202^{d}, the pressure sensors 258a, 258b of delivery apparatus 202^{e} are oriented towards the central longitudinal axis Ca and away from the aortic wall, to remain exposed to the blood flow during the valve implantation procedure and avoid from being contacted by any other tissue that can interfere with such readings.

While two axially spaced pressure sensors 258 are illustrated in FIG. 6, it is to be understood that more than two sensors 258 can optionally be used, for example to increase the resolution of the pressure gradient readings. While two pressure sensors 258a, 258b are coupled to the same elongated positioning member 250 in the illustrated example, it is to be understood that in some examples, such as when a plurality of elongated positioning members 250 are provided, each of the elongated positioning members 250 can include one of the pressure sensors 258, positioned at different axial positions relative to each other.

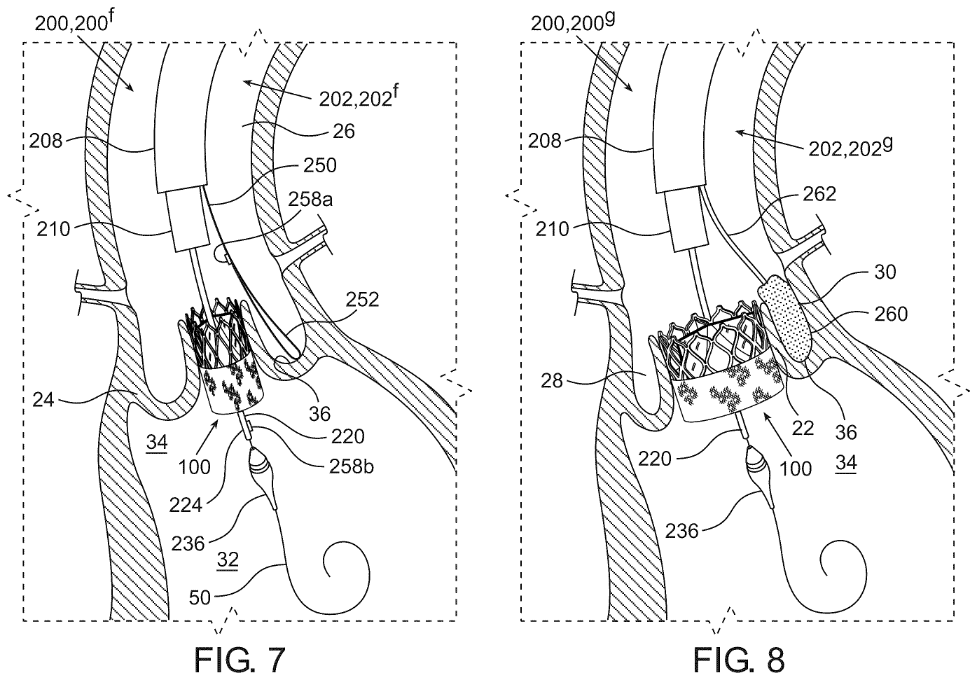

FIG. 7 shows an exemplary delivery assembly 200^{f}. Delivery assembly 200^{f} is an exemplary implementation of delivery assembly 200, and can be similar to any example described above with respect to delivery assembly 200^{e} that includes at least one elongated positioning member 250 having at least one pressure sensor 258 attached thereto, except that instead of at least two laterally-oriented pressure sensors 258 are shown to be coupled to the same positioning member 250 for delivery apparatus 202^{e} in FIG. 6, an elongated positioning member 250 of delivery apparatus 202^{f} can optionally include a pressure sensor 258 (such as a single pressure sensor 258a illustrated in FIG. 7), while another components of the delivery assembly 200^{f} comprises at least one additional pressure sensor 258b.

The first pressure sensor 258a of the delivery assembly 200^{f} is attached to a region of positioning member 250 which is exposed out of the outer delivery shaft 208, yet configured to remain proximal to the outflow end 104 of prosthetic valve 100 during the valve's expansion procedure. The second pressure sensor 258b of the delivery assembly 200^{f} is coupled to another component of the delivery assembly 200, which can be either proximal, distal, or aligned inside of the prosthetic valve 100 during valve implantation procedure. The elongated positioning member 250 and the additional component to which the second pressure sensor 258b is attached can optionally be axially movable relative to each other, such that the first and second pressure sensors 258a, 258b can be axially distanced from each other during valve implantation procedure.

In some examples, the second pressure sensor 258b is coupled to the nosecone shaft 220, such as to a distal portion 224 of the nosecone shaft 220 that can extend past the inflow end 106 of prosthetic valve 100 during the implantation procedure, as illustrated in FIG. 7. Such a configuration allows positioning of the second pressure sensor 258b in the left ventricle 32, such as in the LVOT 34, while the first pressure sensor 258a is positioned in the aorta 26. In this manner, pressure gradients can be measured across the prosthetic valve 100 during expansion and implantation procedure thereof.

While second pressure sensor 258b is illustrated as being coupled to the nosecone shaft 220 in FIG. 7, it is to be understood that this is shown for illustrative purpose, and that second pressure sensor 258b can optionally be similarly coupled to other components of the delivery assembly 200^{f}. In some examples, the second pressure sensor 258b is coupled to the nosecone 236. In some examples, the second pressure sensor 258b is coupled to the balloon catheter 210. In some examples, the second pressure sensor 258b is coupled to the outer delivery shaft 208. In some examples, the second pressure sensor 258b is coupled to the guidewire 50. In some examples, the second pressure sensor 258b is coupled to the prosthetic valve 100 itself, such as to the outer skirt 140, the frame 102, and the like. In some examples, the second pressure sensor 258b is coupled to another shaft of the delivery assembly 200^{f} which is not illustrated in FIG. 7. In some examples, the second pressure sensor 258b is coupled to a pigtail shaft (not shown) of other shaft or catheters that can be used in combination with delivery assembly 200^{f}.

While a single pressure sensor 258a is shown in combination with elongated positioning member 250, and a single pressure sensor 258b is shown in combination with another component of the delivery assembly, such as nosecone shaft 220, are illustrated in FIG. 7, it is to be understood that more than one pressure sensor 258 can optionally be coupled to the elongated positioning member 250, and that more than one sensor 258 can optionally be coupled to the nosecone shaft 220 or any other component of delivery assembly 200^{f}. While only a single other component of delivery assembly 200, such as nosecone shaft 220, is shown to include a pressure sensor 258, it is to be understood that more than one type of additional component of the delivery assembly 200^{f} can optionally include a pressure sensor. For example, a first pressure sensor 258a can optionally be coupled to the elongated positioning member 250, one additional pressure sensor 258b can optionally be coupled to the nosecone shaft 220, and another pressure sensor 258 can optionally be further coupled to another component of the delivery assembly 200, such as to nosecone 236, to prosthetic valve 100, or to any other component.

In some examples, a sensor 258 coupled to the elongated positioning member 250 is a flow sensor 258. The flow sensor 258 can optionally be coupled to a region of positioning member 250 which is exposed out of the outer delivery shaft 208, yet configured to remain proximal to the outflow end 104 of prosthetic valve 100 during the valve's expansion procedure. A flow sensor 258 can be laterally-oriented toward the central longitudinal axis Ca, and can be utilized to measure flow downstream the prosthetic valve 100 during the valve's implantation procedure.

In some examples, the flow sensor 258 comprises an ultrasonic flow sensor. The term “ultrasonic flow sensor”, as used herein, means a flow sensor based on ultrasound detection. Particularly, as known to those skilled in the art, an ultrasonic transducer generates an ultrasonic wave directed at the fluid, and the detected wave after the interaction with fluid indicates the flow velocity of the fluid. In one example, the flow velocity measurement can be performed in any suitable way, such as by measuring a Doppler shift.

In some examples, the flow sensor 258 comprises an optical flow sensor. The term “optical flow sensor”, as used herein, means a flow sensor based on light detection. Particularly, in one example, an optical flow sensor can include a beam of light configured to heat the blood, and fluctuations in temperature caused by variation in the flow are detected by a fiber optic sensor. In some examples, an optical flow sensor can optionally include a pair of light beams and a detection mechanism configured to measure the time difference between the scattering of each of the light beams. In some examples, blood flow can be measured through the use of a monochromatic laser diode. Particularly, the laser probe is inserted into a tissue and turned on, where the light scatters and a small portion is reflected back to the probe. The signal is then processed to calculate the blood flow.

In some examples, the flow sensor 258 comprises a mechanical flow sensor. The term “mechanical flow sensor”, as used herein, means a flow sensor configured to detect flow based on mechanical effects of the fluid flow, as known to those skilled in the art. These mechanical effects can include positive-displacement based flowmeters and/or pressure-based flowmeters.

It is to be understood that one or more elongated positioning member 250 can optionally include any combination of the sensors 258 described above with respect to any of the exemplary delivery assemblies 200^{c}, 200^{d}, 200^{e} and/or 200^{f}. For example, one or more elongated positioning member 250 can include distally-oriented force sensor(s) 258 to identify contact of the elongated positioning member 250 with the proximal abutment surface, laterally-oriented force sensor(s) 258 to measure the force exerted by the prosthetic valve on the surrounding anatomy during expansion thereof, pressure sensor(s) 258 to measure pressure-gradients downstream prosthetic valve 100 and/or across prosthetic valve 100, and/or flow sensor(s) 258 to measure flow downstream prosthetic valve 100.

FIG. 8 shows an exemplary delivery assembly 200^{g}. Delivery assembly 200^{g} is an exemplary implementation of delivery assembly 200, and thus includes all of the features described for delivery assembly 200 throughout the current disclosure, except that the delivery apparatus 202^{g} of delivery assembly 200^{g} further comprises an inflatable positioning balloon 260 at a distal end portion of an inflation tube 262 extending distally from the handle 204. The inflation tube 262 can optionally extend through a shaft or a catheter of the delivery apparatus 202^{g}, such as through a lumen of an outer delivery shaft 208.

In some examples, the inflation tube 262 can optionally extend through a space formed between the balloon catheter 210 and the outer delivery shaft 208 as illustrated in FIG. 8. The inflation tube 262 carrying positioning balloon 260 can optionally be independently maneuvered through a sheath or catheter of the delivery apparatus 202^{g}, such as the outer delivery shaft 208 or any other shaft, and extend out of the shaft (e.g., out of outer delivery shaft 208), for example, via operation of the handle 204 by the clinician. While inflation tube 262 is illustrated to extend out of outer delivery shaft 208, in some examples, the delivery apparatus can optionally include multiple shafts which can be disposed adjacent to each other within the same sheath. Thus, it is to be understood that any reference to an inflation tube and positioning balloon extendable through outer delivery shaft 208 as disclosed herein, can similarly refer to the inflation tube and positioning balloon extending through any other sheath of shaft of the delivery apparatus.

An inflation tube 262 carrying positioning balloon 260 can optionally be axially movable relative to other components of the delivery assembly 200^{g}, such as the prosthetic valve 100, the outer delivery shaft 208, and/or balloon catheter 210. The handle 204 can optionally include a mechanism (not shown) to control axial movement of the positioning balloon 260. While a single inflation tube 262 with positioning balloon 260 is illustrated in FIG. 8, it is to be understood that any other number is contemplated, including any plurality of two or more inflation tubes 262, each having a positioning balloon 260 at its distal end, circumferentially spaced from each other around the prosthetic valve 100.

The inflation tube 262 can optionally be fluidly connectable at a proximal end thereof to a fluid source (not shown) for inflating the positioning balloon 260. The term “inflation fluid”, as used herein, means a fluid (e.g., saline) used for inflating positioning balloon 260. Inflation fluid from the fluid source (e.g., a syringe or a pump) can flow through a lumen of the inflation tube 262 into a cavity of the positioning balloon 260 to inflate the same. Further, the inflation tube 262 may be configured to withdraw fluid from the internal cavity of the positioning balloon 260 to deflate the balloon 260. Positioning balloon 260 is thus configured to transition between a deflated state and an inflated state.

Delivery assembly 200^{g} can optionally be advanced towards the site of implantation, such as a native aortic valve 20, from the ascending aorta 26. The positioning balloon 260 can optionally be retained in a deflated state inside a sheath of the delivery assembly 200^{g}, such as within a lumen of the outer delivery shaft 208, prior to reaching the site of implantation. Upon reaching the aortic valve 20, the inflation tube 262 can optionally be extended from the distal end of outer delivery shaft 208, so as to expose the positioning balloon 260 out of the outer delivery shaft 208 such that the positioning balloon 260 is advanced toward, but optionally spaced from, the proximal abutment surface 36 (e.g., the cusp floor of the native leaflet 22).

At this stage, the positioning balloon 260 can optionally be inflated and advanced further distally so as to an abutment surface at the region of implantation, wherein the distal end of the balloon 260, at the point of contact, is indicative of the position of the respective surface of the native annulus, allowing positioning of the prosthetic valve 100 within the host valve (e.g., native aortic valve) relative to the inflated balloon 260 in a manner similar to that described above with respect to elongated positioning member 250. For example, positioning balloon 260 can optionally be advanced to contact and rest on the proximal abutment surface 36 of the native heart valve 20, and the balloon 260 or inflation fluid injected into its cavity can be radiopaque in a manner that allows identification thereof under fluoroscopy (or other adequate imaging modality), such that the prosthetic valve 100 can be axially moved relative to the bottom end of the balloon 260, indicative of the position of annulus 24, to assist in accurately positioning the prosthetic valve 100 relative to the annulus 24 without the need for injection of a contrast agent.

The inflation tube 262 can optionally be axially movable distal to the outer delivery shaft 208, and can optionally be advanced such that the positioning balloon 260 is spaced radially away from the outer delivery shaft 208 and/or the prosthetic valve 100. Simultaneous with the inflation of balloon 260d, or sequential thereto (e.g., before or after), the prosthetic valve 100 can optionally be advanced from the distal end of the outer delivery shaft 208, for example along with balloon catheter 210, toward and optionally through the native aortic annulus 24, as shown in FIG. 8. The advancement of inflation tube 262 can optionally continue until the positioning balloon 260 reaches the proximal abutment surface 36.

In some examples, when the positioning balloon 260 is in the inflated state, its distal end forms a generally atraumatic surface to avoid damage to the surrounding anatomy during operation. The distal area of the positioning balloon 260 can further increase, in the inflated state, the area of contact with abutment surface 36.

Using tactile feedback from the positioning balloon 260 created by the contact of its distal end with the proximal abutment surface 36, the clinician can axially maneuver the prosthetic valve 100 to position the inflow end 104 at a desired position relative to the surface of the annulus 24 contacted by the balloon 260, in a manner similar to that described above with respect to positioning of a valve 100 relative to an elongate positioning member 250. When the prosthetic valve 100 is positioned at a desired position, relative to the imaged portion of positioning balloon 260, corresponding to the native annulus 24, the clinician can optionally inflate the expansion balloon 212, which can be fed by the same inflation fluid source or a different inflation fluid source, or otherwise (e.g., via removal of a restraining sheath or capsule in the case of self-expandable valve, or actuation of mechanical actuators in the case of mechanically-expandable valves) effectuate expansion/deployment of the prosthetic valve 100 into the desired position within the native annulus 24. During prosthetic valve expansion, the positioning balloon 260 remains engaged with (e.g., pressed against) the proximal abutment surface 36, optionally absorbing at least some of the force exerted by the delivery assembly 200^{g} on aortic valve 20 during the implantation procedure. In some examples, the positioning balloon 260 can optionally be formed to create complaint or semi-compliant structure. The positioning balloon 260 may be formed from PET, Silicone, elastomers, Nylon, Polyethylene or any other polymer of co-polymer.

As mentioned above, in some examples, the balloon can optionally be made of a radiopaque material, or can optionally include radiopaque markings attached thereto. In some examples, the inflation fluid can optionally include fluid contrast media. Any of these solutions will allow visibility of the portion of the balloon 260 contacting the proximal abutment surface 26 during valve positioning, and can also allow tracking of the change in shape of the positioning balloon 260 under fluoroscopy or other suitable imaging modality. As shown in FIG. 8, the positioning balloon 260 is retained between the prosthetic valve 100 and the aortic root inner wall 30, such that prosthetic valve expansion will squeeze the positioning balloon 260 in a manner that can cause appropriate deformation thereof. In some examples, the internal pressure of the inflation fluid within the positioning balloon 260 is low enough to prevent the positioning balloon 260 from resisting valve expansion. In some examples, the inflation tube 262 is configured to allow flow of inflation tube from the positioning balloon 260 back therethrough, to allow gradual balloon deflation as it is being squeezed between the prosthetic valve 100 and the aortic root inner wall 30 during prosthetic valve expansion. In such cases, the position of the frame 102 of prosthetic valve 100 relative to the native anatomy can be visualized and estimated by the deformation of positioning balloon 260.

In some examples, prior to full expansion of the prosthetic valve 100, if the positioning balloon 260 is still at least partially inflated, it can optionally be fully deflated and retracted from the aortic root 28, thereby allowing the prosthetic valve 100 to fully expand against the inner walls 30 of the aortic root 28. The positioning balloon 260 can be optionally (but not necessarily) retracted back into the outer delivery shaft 208, the expansion balloon 212 (if used) can be also deflated to a reduced diameter, and the catheter delivery apparatus 202^{g} can be then withdrawn from the patient, leaving the prosthetic valve 100 implanted within the native valve 20.

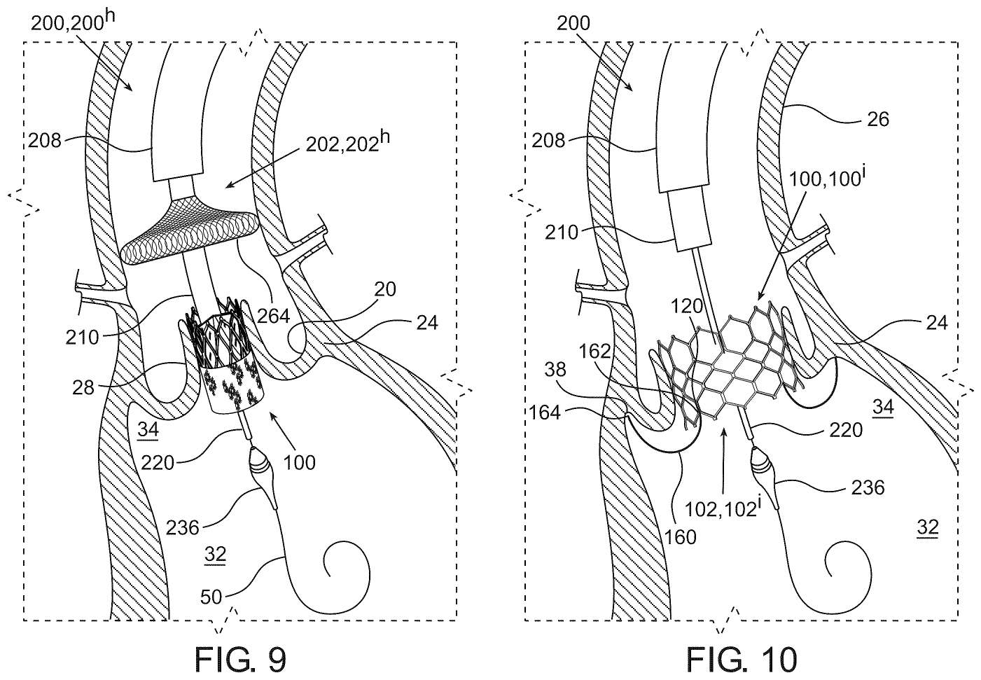

FIG. 9 shows an exemplary delivery assembly 200^{h}. Delivery assembly 200^{h} is an exemplary implementation of delivery assembly 200, and thus includes all of the features described for delivery assembly 200 throughout the current disclosure, except that the delivery apparatus 202^{h} of delivery assembly 200^{h} further comprises a stabilization filter 264. The stabilization filter 264 is configured to serve both as a centering and stabilization component to stabilize the delivery assembly 200^{h} during positioning and expansion of the prosthetic valve 100, and as an embolic filter to capture embolic debris that may dislodge during maneuvering and expanding the prosthetic valve 100 within the native valve 20.

One of the potential complications associated with prosthetic valve implantation is dislodgement of atherosclerotic and/or thrombotic debris, also referred to as “embolic debris”. The most serious consequence of embolic debris is that it travels with the blood flow to downstream vessels and may produce strokes among other maladies of the patient. Advantageously, the disclosed stabilization filter 264 can simultaneously serve both as a stabilization and/or centering member, and as a filter for trapping embolic debris, thereby saving simplifying the delivery assembly 200^{h} and utilization thereof, while also saving costs by replacing two devices that may be utilized separately to provide each of these functionalities, into a single device that provides both functionalities.

The stabilization filter 264 is configured to transition between collapsed and deployed or expanded states. The stabilization filter 264 can optionally comprise a braided mesh, though it can vary in structure and can optionally be, for example, braided, mesh, perforated, and the like. In some examples, the stabilization filter 264 can optionally be constructed from a wire mesh, such as for example a shape-set Nitinol wire braid. The stabilization filter 264 can optionally define pores sized o optimize embolic capture of blood flowing therethrough. In some examples, the average or maximal pore size of the stabilization filter 264 can optionally be 40, 100, 150, 200, or 300 microns, or any value in between.

In some examples, the stabilization filter 264 can optionally be independently maneuvered through a sheath or catheter of the delivery apparatus 200^{h}, such as the outer delivery shaft 208 or any other shaft, and deployed out of the shaft (e.g., out of outer delivery shaft 208), for example, via operation of the handle 204 by the clinician.

Delivery assembly 200^{h} can optionally be advanced towards the site of implantation, such as a native aortic valve 20, from the ascending aorta 26. The stabilization filter 264 can optionally be retained in a collapsed state inside a sheath of the delivery assembly 200^{h}, such as within a lumen of the outer delivery shaft 208, prior to reaching the site of implantation. Upon reaching the aortic valve 20, the stabilization filter 264 can optionally be deployed from the distal end of outer delivery shaft 208, exposing it out of the outer delivery shaft 208 such that the stabilization filter 264 can optionally self-expand radially against the aortic wall, at a position which is proximal to the aortic annulus 24, in a manner that allows the prosthetic valve 100 or any shaft or catheter coupled to or carrying prosthetic valve 100 to distally extend therethrough towards the native annulus 24. The pores of the stabilization filter 264 allow blood flow therethrough during the implantation procedure.

In its deployed and expanded state, the stabilization filter 264 can serve as a docking or positioning member configured to circumferentially center the prosthetic valve 100 and/or a catheter/shaft attached thereto, such as balloon catheter 210, to improve accuracy of prosthetic valve 100 deployment and implantation. As mentioned above, the stabilization filter 264 can optionally be formed of a shape-memory material configured to self-expand when not restricted by an outer shaft, such as outer delivery shaft 208. Shape memory can be imparted to a braided mesh by thermal treatment to achieve a spring temper in stainless steel, for example, or to set a shape memory in a susceptible metal alloy, such as Nitinol. When the stabilization filter 264 is expanded against the aortic wall, the force applied by the stabilization filter 264 against the surrounding anatomy is sufficiently high to retain it in position and resist unintentional axial movement thereof during maneuvering and expansion of the prosthetic valve 100.

In some examples, the stabilization filter 264 is attached to a shaft of the delivery assembly 200^{h}, which can optionally be axially movable with respect to an external sheath or shaft, such as the outer delivery shaft 208. In some examples, the stabilization filter 264 is coupled to a portion of the balloon catheter 210, proximal to the expansion balloon 212, such that upon reaching the site of implantation, pushing the balloon catheter 210 distally relative to the outer delivery shaft 208, and/or retracting the outer delivery shaft 208 relative to the balloon catheter 210, serves to expose the stabilization filter 264 and allow expansion thereof against the aortic wall, as shown in FIG. 9. In some examples, the stabilization filter 264 can optionally be coupled to a different shaft passable through the outer delivery shaft 208, such as a push shaft 214 or other independent shaft (not illustrated).

In order to prevent embolic debris from flowing downstream past stabilization filter 264, the stabilization filter 264 extends across the entire cross-sectional area of the blood vessel (e.g., aorta 26) between the blood vessel's wall and a shaft or catheter it is attached to, such that no openings exist, in its expanded state, between the outermost diameter of the stabilization filter 264 and the region of attachment to the shaft carrying it, larger than the pore size defined by the mesh. In this manner, as long as the stabilization filter 264 is expanded proximally to the prosthetic valve 100, embolic debris suspended in the blood are captured by the stabilization filter 264 to prevent them from flowing into distal vessel beds.

Simultaneous with the deployment of stabilization filter 264, or sequential thereto (e.g., before or after), the prosthetic valve 100 can optionally be advanced from the distal end of the outer delivery shaft 208, for example along with balloon catheter 210, toward and optionally through the native aortic annulus 24, as shown in FIG. 9. With the stabilization filter 264 retained in position, the clinician can optionally inflate the expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100 into the desired position within the native annulus 24. Optionally. after full or at least partial expansion of the prosthetic valve 100, re-sheathing of the stabilization filter 264, such as back into the outer delivery shaft 208, can be performed, the expansion balloon 212 (if used) can be deflated to a reduced diameter, and the catheter delivery apparatus 202^{h} can be then withdrawn from the patient, leaving the prosthetic valve 100 implanted within the native valve 20.

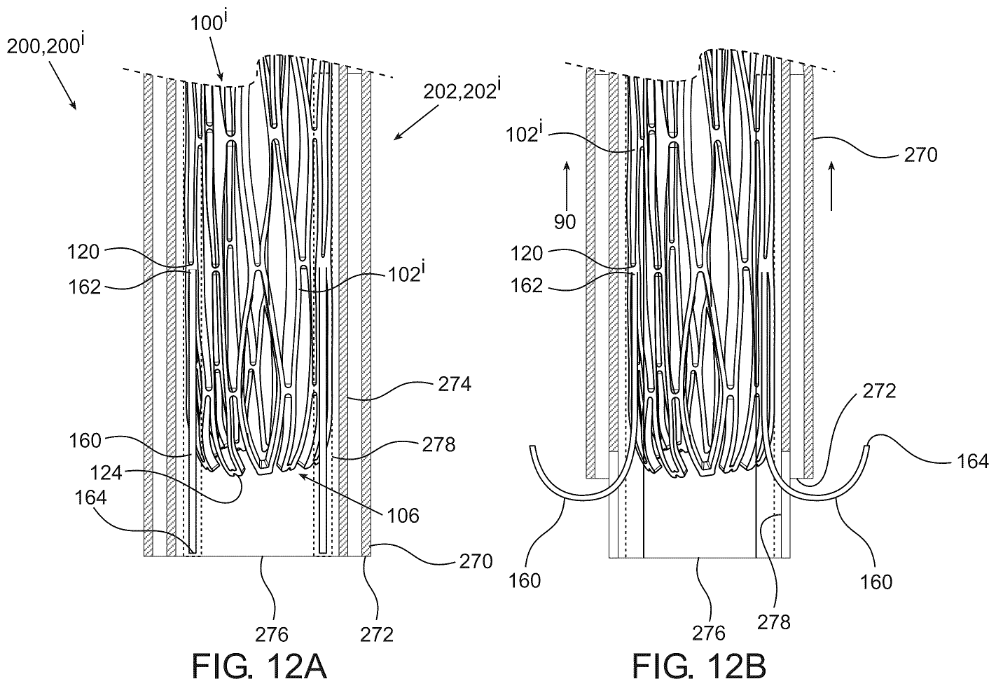

FIG. 10 shows a delivery assembly 200 carrying an exemplary prosthetic valve 100ⁱ, deployed within a native aortic valve 20. Prosthetic valve 100ⁱ is an exemplary implementation of prosthetic valve 100, and thus includes all of the features described for prosthetic valve 100 throughout the current disclosure, except that the prosthetic valve 100ⁱ further comprises one or more positioning struts 160, configured to transition between a compacted state and a deployed or free state. Prosthetic valve 100ⁱ is shown in FIG. 10, as well as in FIGS. 12A-12B described below, without soft components such as skirts or leaflets, for the sake of clarity.

Each positioning strut 160 has a fixed end 162 attached to the frame 102, and a free end 164 opposite to the fixed end 162. Any reference to “positioning struts 160” in the plural form herein, can similarly refer to a single positioning struts 160, unless stated otherwise. In some examples, the positioning struts 160 are integrally formed with the remainder of the frame 102. For example, if the frame 102 is formed by laser cutting a single tube, positioning struts 160 may also be formed by laser cutting the same tube. In some examples, the positioning struts 160 are formed separately and then attached to frame 102, for example by adhesives, sutures, welding, or otherwise.

In some examples, the positioning struts 160 are coupled, at their fixed ends 162, to junctions 120 of the frame 102, which can optionally be closer to the inflow end 106 than to the outflow end 104, as illustrated in FIG. 10. In some examples, the positioning struts 160 are coupled, at their fixed ends 162, to the inflow apices 124. In some examples, the free ends 164 are constructed to be atraumatic (e.g., blunt or otherwise lacking sharp edges) to avoid damage to the surrounding anatomy during operation, such as by being rounded, covered by atraumatic covering, coated, and the like.

The positioning struts 160 can optionally be shape-set, for example by thermal treatment, so that when not constrained by an outer covering element, such as a sheath or a capsule, the positioning struts 160 extend radially away from the central longitudinal axis Ca, such that the free ends 164 are positioned radially away from the frame 102, and are proximally-oriented, in the free or deployed state.

The native valve 20 can define a distal abutment surface 38, defined as a distally facing surface of the native valve 20, opposite to the proximal abutment surface 36. The distal abutment surface 38 can face the LVOT 34. Delivery assembly 200 can be advanced towards the site of implantation, such as a native aortic valve 20, from the ascending aorta 26. The prosthetic valve 100 can optionally be retained in a crimped or compressed configuration prior to reaching the site of implantation, with the positioning struts 160 also being similarly retained in a compacted state, optionally extending in a relatively straight manner (e.g., parallel to central longitudinal axis Ca), when the valve 100ⁱ is positioned inside a capsule or a sheath (such as outer delivery shaft 208). The valve is advanced such that an inflow portion thereof resides within the left ventricle 32, at which point the prosthetic valve 100ⁱ can optionally be exposed from the sheath or capsule by being distally advanced relative thereto, and/or by retracting the sheath or capsule from the valve 100ⁱ.

This allows the positioning struts 160 to spring into their free or deployed pre-formed state, optionally curving radially outward away from the frame 102, such that the free ends 164 are proximally directed towards the annulus 24 and the distal abutment surface 38. If the free ends 164 are distally spaced from the distal abutment surface 38, the prosthetic valve 100ⁱ can optionally be proximally pulled to approximate the positioning struts 160 to the annulus 24, such that the free ends 164 are brought into contact with the distal abutment surface 38. In some examples, the prosthetic valve 100 can optionally be at least partially expanded simultaneous with, of sequentially to, proximally pulling it relative to annulus 24, which can help place the free ends 164 radially farther away, to align with the distal abutment surface 38. It is to be understood that while two positioning struts 160 are illustrated in FIG. 10, any other number is contemplated, such as a single positioning strut 160 or more than two positioning struts 160.

Using tactile feedback from the positioning struts 160 created by the contact of the free ends 164 with the distal abutment surface 38, the clinician can axially maneuver the prosthetic valve 100ⁱ to position the inflow end 104 at a desired position relative to the surface of the annulus 24 contacted by the positioning struts 160, in a manner similar to that described above with respect to positioning of a valve 100 relative to an elongate positioning member 250. When the prosthetic valve 100ⁱ is positioned at a desired position, relative to the imaged portion of positioning struts 160, corresponding to the native annulus 24, the clinician can optionally inflate an expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100ⁱ within the native annulus 24. During prosthetic valve expansion, the positioning struts 160 remain engaged with (e.g., pressed against) the distal abutment surface 38, optionally absorbing at least some of the force exerted by the delivery assembly 200 on aortic valve 20 during the implantation procedure.

In some examples, the positioning struts 160 can be relatively conspicuous under applicable image-guidance modalities, such as fluoroscopic, echocardiographic, and/or other imaging methods. As mentioned above, the positioning struts 160 can optionally be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the positioning struts 160 are either made of a radiopaque material or comprise radiopaque markers. This can assist, in some cases, in identification of contact of the positioning struts 160 with the abutment surface 38. For example, when using fluoroscopy or other appropriate imaging technique, the clinician can see the positioning struts 160 begin to buckle, bend or otherwise deform in shape, when engaged with (i.e., pressed against) the distal abutment surface 38. Thus, contact of the positioning struts 160 can be identified by tactile feedback and/or visually from fluoroscopy or other image guidance.

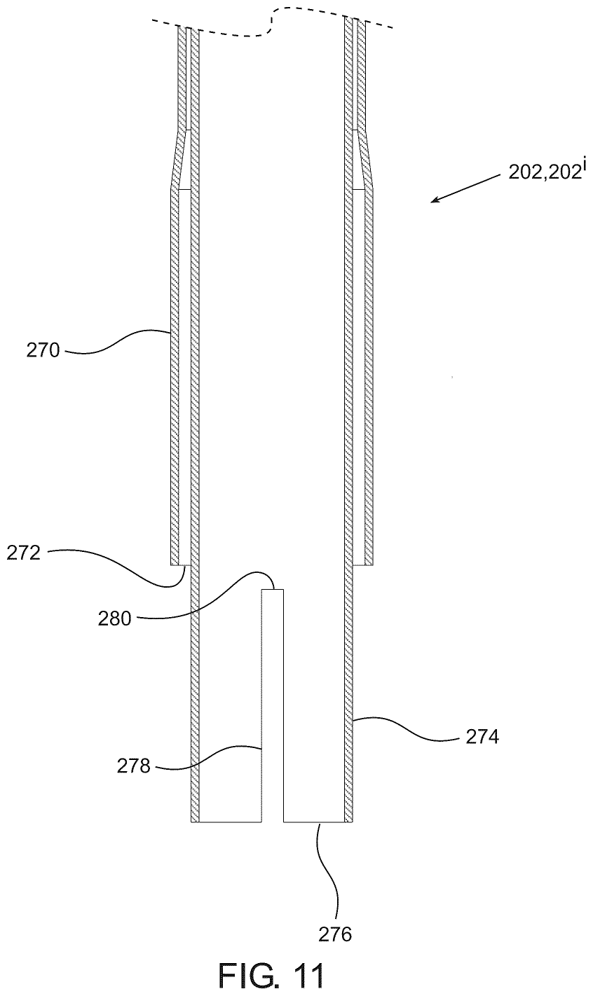

FIGS. 11-12B show a distal portion of an exemplary delivery assembly 200ⁱ that can be utilized for delivery and deployment of prosthetic valve 100ⁱ. Delivery assembly 200ⁱ is an exemplary implementation of delivery assembly 200, and thus includes all of the features described for delivery assembly 200 throughout the current disclosure, except that the delivery apparatus 202ⁱ of delivery assembly 200ⁱ further comprises an inner capsule 274 and an outer capsule 270 axially movable relative to each other.

FIG. 11 shows the inner capsule 274 disposed inside the outer capsule 270, with the prosthetic valve 100ⁱ or other components of the delivery apparatus 202ⁱ removed from view for clarity. Each of the inner capsule 274 and the outer capsule 270 can optionally be formed at distal portions of corresponding shafts (not shown), that can optionally extend inside another sheath of shaft of the delivery apparatus 202ⁱ, such as the outer delivery shaft 208. For example, the outer capsule 270 and/or a shaft attached thereto can extend through a lumen of the outer delivery shaft 208. The outer capsule 270 defines a diameter that is greater than the diameter defined by the inner capsule 274, such that the inner capsule 274 can reside inside of, and extend through, the outer capsule 270.

It is to be understood that an outer capsule 270 can be a separate component attached to a corresponding shaft, or can be formed as an integral distal portion of a shaft. Similarly, an inner capsule 274 can be a separate component attached to a corresponding shaft, which can extend through a lumen of the shaft of the outer capsule, or can be formed as an integral distal portion of a shaft.

The inner capsule 274 extends proximally from an inner capsule distal end 276, and the outer capsule 270 extends proximally from an outer capsule distal end 272. In some examples, the inner capsule 274 can optionally further include one or more slots 278, each slot 278 extending from the inner capsule distal end 276 to a slot proximal end 280. The number of slots 278 can optionally match the number of positioning struts 160 of a valve 100ⁱ configured to reside inside inner capsule 274. The position of the slots 278 around the circumference of the inner capsule 274 can optionally match the position of the positioning struts 160 around the circumference of frame 102. The width of each slot 278 can optionally be greater than the width of positioning strut 160 to allow movement thereof through the slot 278. The length of each slot 278 can be set to allow appropriate extension of the positioning strut 160 as it passes therethrough when transitioning between the collapsed and free states. In the example illustrated in FIG. 11, the inner capsule 274 is shown partially offset distally to the outer capsule 270, so as to expose the entire length of the slot 278 out of outer capsule 270.

FIGS. 12A and 12B show two states of the distal portion of the delivery apparatus 202ⁱ, showing the positioning struts 160 in a compacted state (FIG. 12A) and free state (FIG. 12B), while the prosthetic valve 100ⁱ is still retained in a compressed configuration. FIG. 12A shows the state of the prosthetic valve 100ⁱ and the positioning struts 160 during delivery toward the site of implantation. As shown, in this state, the prosthetic valve 100 is retained in a crimped or compressed configuration inside inner capsule 274, while the outer capsule 270 is disposed around and covers the inner capsule 274. The outer capsule 270 can optionally be aligned with the inner capsules 274 in this state, such that the outer capsule distal end 272 is aligned with, or in close proximity to, the inner capsule distal end 276, though this is not mandatory, as long as the outer capsule 270 covers a sufficient portion of the slots 278 to retain the positioning struts 160 in the compacted state.

As shown, when the prosthetic valve 100ⁱ is positioned inside inner capsule 274, the positioning struts 160 are aligned with slots 278, and strive to extend outward through the slots 278. The inner wall of the outer capsule 270 can optionally retain the positioning struts 160 in a relatively straight configuration, as illustrated in FIG. 12A. When the distal portion of the prosthetic valve 100ⁱ, including positioning struts 160, is inside the left ventricle 32, such as optionally inside the LVOT 34, the outer capsule 270 can be retracted in a proximal direction 90 relative to the inner capsule 274, and/or the inner capsule 274 can be distally pushed relative to the outer capsule 270, such that the outer capsule distal end 272 can optionally be placed at or proximal to the slot proximal ends 280, exposing the slots 278, and allowing the positioning struts 160 to spring radially outwards and assume their pre-shaped free state, as shown in FIG. 12B. The positioning struts 160 can optionally be pre-shaped to assume a curved C-shaped or U-shaped configuration in their free state, concave at the sides facing the distal abutment surface 38, as illustrated.

Both inner capsule 274 and outer capsule 270 can be then proximally retracted from the prosthetic valve 100ⁱ to expose the prosthetic valve 100ⁱ, so as to allow expansion thereof as described above with respect to FIG. 10.

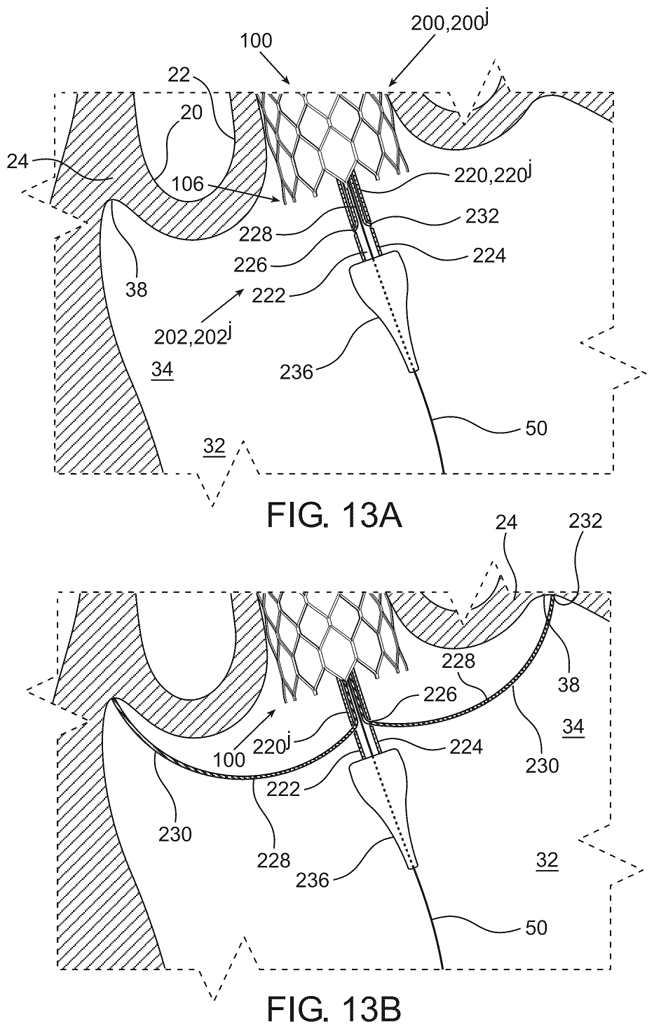

FIGS. 13A-13B show an exemplary delivery assembly 200^{j}. Delivery assembly 200^{j} is an exemplary implementation of delivery assembly 200, and thus includes all of the features described for delivery assembly 200 throughout the current disclosure, except that the delivery apparatus 202^{j} of delivery assembly 200^{j} further comprises one or more positioning arms 228 extending through the lumen 222 of nosecone shaft 220J, and configured to transition between a compacted state and a deployed or free state. Prosthetic valve 100 is shown in FIGS. 13A-13B without soft components such as skirts or leaflets, for the sake of clarity.

Any reference to “positioning arms 228” in the plural form herein, can similarly refer to a single positioning arm 228, unless stated otherwise. The nosecone shaft 220J comprises one or more side openings 226 formed at a distal portion 224 thereof, for example proximal to nosecone 236. The positioning arms 228 are axially movable relative to nosecone shaft 2201, such as toward and away from the side openings 226. The number of side openings 226 can optionally match the number of positioning arms 228. Each positioning arm 228 defines a free end 232 that can optionally extend through the corresponding side opening 226. The proximal ends of the positioning arms 228 (not shown) can optionally extend to the handle 204, and be operated by a knob 206 to apply axially move the positioning arms 228 in the distal or proximal directions.

The positioning arms 228 can optionally be shape-set, for example by thermal treatment, so that when not constrained by the wall of nosecone shaft 220ⁱ as shown in FIG. 13A, the positioning arms 228 can optionally extend radially away from the central longitudinal axis Ca, such that the free ends 232 are positioned radially away from the nosecone shaft 220J, and are proximally-oriented, in the free or deployed state.

Delivery assembly 200^{j} can optionally be advanced towards the site of implantation, such as a native aortic valve 20, from the ascending aorta 26. The positioning arms 228 can optionally be retained in a compacted state prior to reaching the site of implantation, optionally extending in a relatively straight manner (e.g., parallel to central longitudinal axis Ca), with the free ends 232 aligned with, or positioned proximal to, the side openings 226, as shown in FIG. 13A. The prosthetic valve 100 is advanced such that an inflow portion thereof resides within the left ventricle 32, and the nosecone shaft distal portion 224 extends past the valve 100 such that the side openings 226 are positioned distal to the inflow end 106 of the valve 100.

At this stage, as shown in FIG. 13B, the positioning arms 228 can optionally be distally advanced through the nosecone shaft lumen 222, such that the positioning arms 228 extend through the side openings 226 radially away from the nosecone shaft 220J, assuming their pre-shaped free state, forming curved section 230 between the side openings 226 and the free ends 232, with the free ends 232 directed towards the annulus 24 and the distal abutment surface 38. The clinician can control the length of the exposed portion of the positioning arms 228, to form longer or shorter curved sections 230 according to a patient-specific anatomy, so as to align the free ends 232 with the distal abutment surface 38. It is to be understood that while two positioning arms 228 are illustrated in FIGS. 13A-13B, any other number is contemplated, such as a single positioning arm 228 or more than two positioning arms 228.

Using tactile feedback from the positioning arms 228 created by the contact of the free ends 232 with the distal abutment surface 38, the clinician axially maneuver the prosthetic valve 100 to position the inflow end 104 at a desired position relative to the surface of the annulus 24 contacted by the positioning arms 228, in a manner similar to that described above with respect to positioning of a valve 100 relative to an elongate positioning member 250. When the prosthetic valve 100 is positioned at a desired position, relative to the imaged portion of positioning arms 228, corresponding to the native annulus 24, the clinician can optionally inflate an expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100 within the native annulus 24. The positioning arms 228 can optionally be pre-shaped to form C-shaped or U-shaped curved sections 230 in their free state, concave at the sides facing the distal abutment surface 38, as illustrated.

During prosthetic valve expansion, the positioning arms 228 remain engaged with (e.g., pressed against) the distal abutment surface 38, optionally absorbing at least some of the force exerted by the delivery assembly 200^{j} on aortic valve 20 during the implantation procedure.

In some examples, the positioning arms 228 can be relatively conspicuous under applicable image-guidance modalities, such as fluoroscopic, echocardiographic, and/or other imaging methods. As mentioned above, the positioning arms 228 can optionally be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the positioning arms 228 are either made of a radiopaque material or comprises radiopaque markers. This can assist, in some cases, in identification of contact of the positioning arms 228 with the abutment surface 38. For example, when using fluoroscopy or other appropriate imaging technique, the clinician can see the positioning arms 228 begin to buckle, bend or otherwise deform in shape, when engaged with (i.e., pressed against) the distal abutment surface 38. Thus, contact of the positioning arms 228 can be identified by tactile feedback and/or visually from fluoroscopy or other image guidance.

In some examples, prior to full expansion of the prosthetic valve 100, the positioning arms 228 can optionally be retracted back into the nosecone shaft lumen 222. Optionally, after full expansion of the prosthetic valve 100, the balloon 212 (if used) can be deflated to a reduced diameter, and the catheter delivery apparatus 202^{j} can be then withdrawn from the patient, leaving the prosthetic valve 100 implanted within the native valve 20.

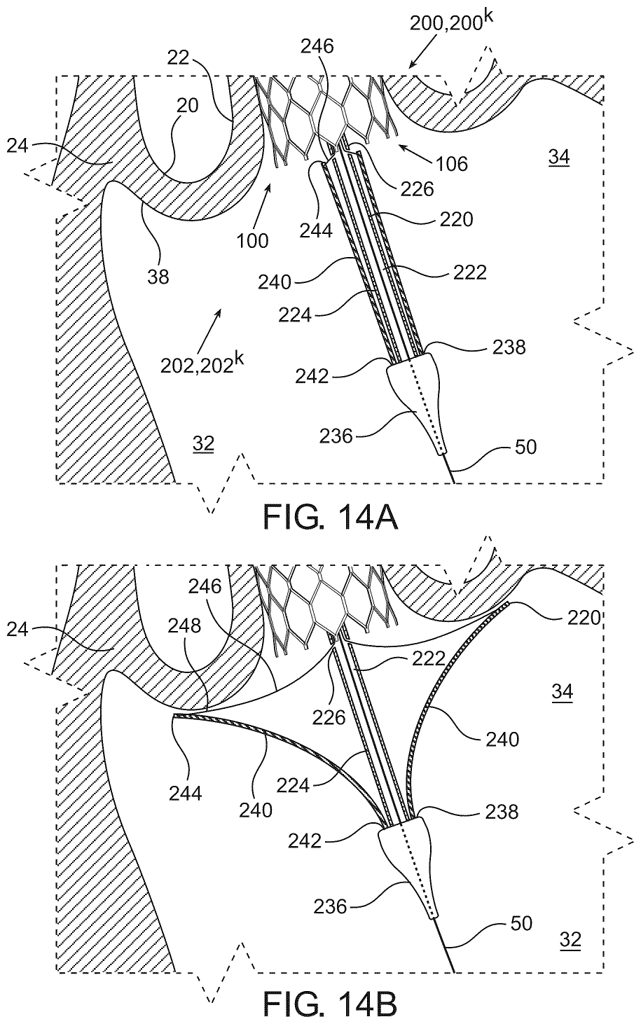

FIGS. 14A-14B show an exemplary delivery assembly 200^{k}. Delivery assembly 200^{k} is an exemplary implementation of delivery assembly 200, and thus includes all of the features described for delivery assembly 200 throughout the current disclosure, except that the delivery apparatus 202^{k} of delivery assembly 200^{k} further comprises one or more positioning arms 240 extending proximally from the nosecone 236, and configured to transition between a compacted state and a deployed or free state. Prosthetic valve 100 is shown in FIGS. 14A-14B without soft components such as skirts or leaflets, for the sake of clarity.

Any reference to “positioning arms 240” in the plural form herein, can similarly refer to a single positioning arm 240, unless stated otherwise. Each positioning arm 240 includes a fixed end 242 at which it is attached to the nosecone 236, such as by gluing, welding, suturing, and the like, and an opposite free end 244. In some examples, the positioning arms 240 are coupled, at their fixed ends 242, to a proximal end 238 of the nosecone, as illustrated.

Tensioning members 246 or tethers can optionally be coupled to the positioning arms 240. For example, distal ends 248 of the tensioning member can optionally be attached, such as by being tied, glued, or otherwise coupled, to the free ends 244 of the positioning arms 240. The tensioning members 246 or tethers can optionally be in the form of pull wires, cables, strings, sutures, and the like. The number of tensioning members 246 can optionally match the number of positioning arms 240. The proximal ends of the tensioning members 246 (not shown) can optionally extend to the handle 204, and be operated by a knob 206 to apply tension thereto or release tension therefrom. When the tensioning members 246 are tensioned, as shown in FIG. 14A, the positioning arms can be forced to assume a relatively straight configuration, substantially parallel to the central longitudinal axis Ca, and/or parallel to the nosecone shaft 220, as shown in FIG. 14A.

The positioning arms 240 can optionally be shape-set, for example by thermal treatment, so that when tension is released from the tensioning members 246, the positioning arms 240 can spring radially away from the central longitudinal axis Ca, such that the free ends 244 are positioned radially farther away from the nosecone shaft 220, and can be generally laterally and proximally-oriented, in the free or deployed state.

Delivery assembly 200^{k} can optionally be advanced towards the site of implantation, such as a native aortic valve 20, from the ascending aorta 26. The tensioning members can be kept taut during delivery, approximating the free ends 244 toward the nosecone shaft 220 to retain the positioning arms 240 in a relatively straight compacted state prior to reaching the site of implantation (e.g., parallel to central longitudinal axis Ca and/or to the nosecone shaft distal portion 224), as shown in FIG. 14A. The prosthetic valve 100 is advanced such that an inflow portion thereof resides within the left ventricle 32, and the nosecone shaft distal portion 224 extends past the valve 100 such that the free ends 244 of positioning arms 240 are positioned distal to the inflow end 106 of the valve 100.

At this stage, as shown in FIG. 14B, tension can optionally be released from the tensioning members 246, such that the positioning arms 240 can spring radially outwards to their pre-shaped free state, with the free ends 244 positioned father away from the nosecone shaft 220. The clinician can control the tension of the tensioning members 246, optionally releasing only some of the tension, sufficient to align the free ends 244 with the distal abutment surface 38. It is to be understood that while two positioning arms 240 are illustrated in FIGS. 14A-14B, any other number is contemplated, such as a single positioning arm 240 or more than two positioning arms 240.

Using tactile feedback from the positioning arms 240 created by the contact of the free ends 244 with the distal abutment surface 38, the clinician can axially maneuver the prosthetic valve 100 to position the inflow end 104 at a desired position relative to the surface of the annulus 24 contacted by the free ends 244 of positioning arms 240, in a manner similar to that described above with respect to positioning of a valve 100 relative to an elongate positioning member 250. When the prosthetic valve 100 is positioned at a desired position, relative to the imaged portion of the free ends 244 of positioning arms 240, corresponding to the native annulus 24, the clinician can optionally inflate an expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100 within the native annulus 24. The positioning arms 240 can optionally be pre-shaped to assume an arcuate shape in the free state, as illustrated.

During prosthetic valve expansion, the positioning arms 240 remain engaged with (e.g., pressed against) the distal abutment surface 38, optionally absorbing at least some of the force exerted by the delivery assembly 200^{k} on aortic valve 20 during the implantation procedure.

In some examples, the positioning arms 240 can be relatively conspicuous under applicable image-guidance modalities, such as fluoroscopic, echocardiographic, and/or other imaging methods. As mentioned above, the positioning arms 240 can optionally be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the positioning arms 240 are either made of a radiopaque material or comprises radiopaque markers. This can assist, in some cases, in identification of contact of the positioning arms 240 with the abutment surface 38. For example, when using fluoroscopy or other appropriate imaging technique, the clinician can see the positioning arms 240 begin to buckle, bend or otherwise deform in shape, when engaged with (i.e., pressed against) the distal abutment surface 38. Thus, contact of the positioning arms 240 can be identified by tactile feedback and/or visually from fluoroscopy or other image guidance.

In some examples, prior to full expansion of the prosthetic valve 100, tension can optionally be re-applied to the tensioning members 246 to force the tensioning members to re-assume the compacted configuration. The nosecone 236 can be optionally translated distally prior to re-tensioning the tensioning members 246. Optionally, after full expansion of the prosthetic valve 100, the balloon 212 (if used) can be deflated to a reduced diameter, and the catheter delivery apparatus 202^{k} can be then withdrawn from the patient, leaving the prosthetic valve 100 implanted within the native valve 20.

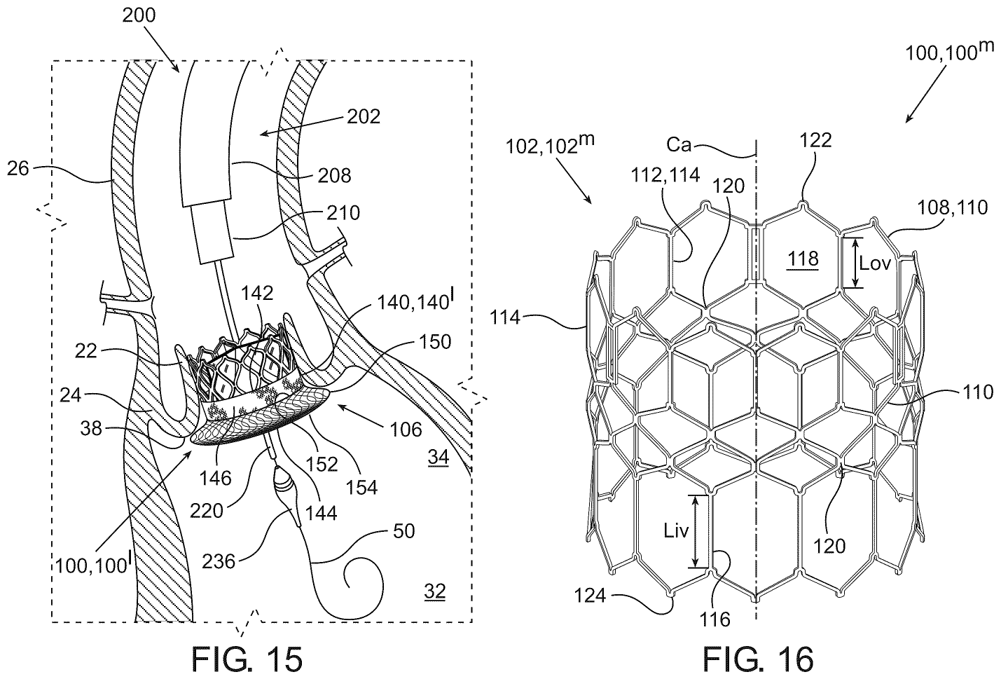

FIG. 15 shows a delivery assembly 200 carrying an exemplary prosthetic valve 100^{l}, deployed within a native aortic valve 20. Prosthetic valve 100^{l} is an exemplary implementation of prosthetic valve 100, and thus includes all of the features described for prosthetic valve 100 throughout the current disclosure, except that an outer skirt 1501 of the prosthetic valve 100^{l} further comprises a circumferential mesh 150, configured to transition between a compacted state and an expanded free state.

The outer skirt 150^{l} can optionally include a base layer 146 extending from the outer skirt inflow end 144 to the outer skirt outflow end 142, with the circumferential mesh 150 coupled, such as by being sutured or otherwise attached, to the base layer 146. The base layer 146 can optionally be coupled (e.g., sutured) to the frame 102. The circumferential mesh 150 can extend from a mesh distal end 154 to a mesh proximal end 152. In some examples, the mesh distal end 154 is aligned with, or positioned proximate to, the outer skirt inflow end 144, while the mesh proximal end 152 can optionally be distal to the outer skirt outflow end 142. In some examples, the circumferential mesh 150 extends around an inflow portion of the frame 102, such that the mesh distal end 154 is aligned with, or positioned proximate to, the inflow end 106.

The circumferential mesh 150 can optionally comprise a braided, flexible material. The circumferential mesh 150 can optionally be formed of a shape-memory material, such as Nitinol, and pre-shaped so that when not constrained by an outer covering element, such as a sheath or a capsule, the circumferential mesh 150 extends radially away from the frame 102.

Delivery assembly 200 can optionally be advanced towards the site of implantation, such as a native aortic valve 20, from the ascending aorta 26. The prosthetic valve 100^{l} can optionally be retained in a crimped or compressed configuration prior to reaching the site of implantation, with the circumferential mesh 150 also being retained in a compacted state, generally flattened between the frame 102 and a capsule or a sheath (such as outer delivery shaft 208, or such as any of the capsules 270, 274 described above) in which the valve 100^{l} is retained. The valve is advanced such that an inflow portion thereof resides within the left ventricle 32, at which point the prosthetic valve 100^{l} can be exposed from the sheath or capsule by being distally advanced relative thereto, and/or by retracting the sheath or capsule from the valve 100^{l}.

This allows the circumferential mesh 150 to spring into its free pre-formed state, expanded radially outward away from the frame 102. The outer diameter of the circumferential mesh 150 in its free state is set to position a sufficient portion of the circumferential mesh 150 below distal abutment surface 38, such that when contacting the abutment surface 38, an appropriate portion of the circumferential mesh 150 will resist further proximal displacement of the prosthetic valve 100^{l} through the annulus 24. In some examples, the radial distance assumed by the circumferential mesh 150 in its expanded free state, between the frame 102 (or the base layer 146) and the outermost edge of the circumferential mesh, is greater than half the diameter of the frame 102 in it expanded configuration.

In some examples, the radial distance assumed by the circumferential mesh 150 in its expanded free state is at least as great as the diameter of the frame 102 in its expanded configuration. The radial distance to which the circumferential mesh 150 can expand is selected to be large enough such that when the prosthetic valve 100^{l} is released from the capsule (or sheath), sufficient contact area will be provided by the circumferential mesh 150, so as to contact the distal abutment surface 38, while the prosthetic valve 100^{l} is still fully or at least partially compressed. If the outermost edge of the circumferential mesh 150 contact and is pressed against side walls of the left ventricle 32 as the prosthetic valve is expanded, the circumferential mesh 150 can be radially squeezed between the frame 102 and the surrounding anatomical walls, allowing the circumferential mesh 150 to assume a narrower radial distance between the frame and its outermost edge, without interfering with prosthetic valve expansion.

If the circumferential mesh 150 is distally spaced from the distal abutment surface 38, the prosthetic valve 100^{l} can optionally be proximally pulled to approximate the circumferential mesh 150 to the annulus 24, such that a proximal surface of the circumferential mesh 150 is brought into contact with the distal abutment surface 38. In some examples, the prosthetic valve 100^{l} can optionally be at least partially expanded simultaneous with, of sequentially to, proximally pulling it relative to annulus 24, which can help place the circumferential mesh 150 radially farther away, to align with the distal abutment surface 38.

Using tactile feedback from the circumferential mesh 150 created by its contact with the distal abutment surface 38, the clinician can axially maneuver the prosthetic valve 100^{l} to position the inflow end 104 at a desired position relative to the surface of the annulus 24 contacted by the circumferential mesh 150, in a manner similar to that described above with respect to positioning of a valve 100 relative to an elongate positioning member 250. When the prosthetic valve 100^{l} is positioned at a desired position, relative to the imaged circumferential mesh 150, corresponding to the native annulus 24, the clinician can optionally inflate an expansion balloon 212 or otherwise (e.g., via removal of a restraining sheath or capsule in the case of self-expandable valve, or actuation of mechanical actuators in the case of mechanically-expandable valves) effectuate expansion/deployment of the prosthetic valve 100^{l} within the native annulus 24. During prosthetic valve expansion, the positioning struts 160 remain engaged with (e.g., pressed against) the distal abutment surface 38, optionally absorbing at least some of the force exerted by the delivery assembly 200 on aortic valve 20 during the implantation procedure.

In some examples, the circumferential mesh 150 can be relatively conspicuous under applicable image-guidance modalities, such as fluoroscopic, echocardiographic, and/or other imaging methods. As mentioned above, the circumferential mesh 150 can optionally be made, in some examples, of a shape memory material such as Nitinol. As Nitinol is sometime hard to see on fluoroscopy, radiopaque markers (such as gold), could be added, or the Nitinol could be mixed with a radiopaque material for easy identification on fluoroscopy during the procedure. Thus, in some examples, the circumferential mesh 150 is either made of a radiopaque material or comprises radiopaque markers. This can assist, in some cases, in identification of contact of the circumferential mesh 150 with the abutment surface 38. For example, when using fluoroscopy or other appropriate imaging technique, the clinician can see the circumferential mesh 150 begin to axially compress or otherwise deform in shape, when engaged with (i.e., pressed against) the distal abutment surface 38. Thus, contact of the circumferential mesh 150 can be identified by tactile feedback and/or visually from fluoroscopy or other image guidance.

Some of the positioning members and solutions disclosed herein are designed to have tips or contact surfaces (configured to contact an abutment surface such as surface 36 or 38) at a certain axial distance from the inflow end 106 of the prosthetic valve 100, such that when pressed against an abutment surface, the inflow end 106 remains at a desired axial position relative to the annulus 24. When a prosthetic valve radially expands, it usually foreshortens in length between the inflow and outflow ends. A conventional prosthetic valve, such as prosthetic valve 100^{a} illustrated in FIGS. 1A-1B, conventionally foreshortens such that the inflow end 106 axially moves towards the outflow end 104, which can result from the higher rigidity at the outflow region of the frame 102^{a} due to the longer outflow vertical struts 114.

FIG. 16 shows a frame 102^{m} of an exemplary prosthetic valve 100^{m}. Prosthetic valve 100^{m} is an exemplary implementation of prosthetic valve 100, and thus includes all of the features described for prosthetic valve 100 throughout the current disclosure, except that the frame 102^{m} of prosthetic valve 100^{m} comprises inflow vertical struts 116 which are the longest vertical struts 112 of the frame. Prosthetic valve 100^{m} is shown in FIG. 16 without soft components such as skirts or leaflets, for the sake of clarity.

In some examples, vertical struts 112 of frame 102^{m} comprise outflow vertical struts 114 having a length Lov and inflow vertical struts 116 having a length Liv, such that the length Liv of the inflow vertical struts 116 is greater than the length Lov of the outflow vertical struts 114 (i.e., Liv>Lov), as illustrated in FIG. 16. The longer inflow vertical struts 116 will result in greater overall rigidity of the inflow portion of prosthetic valve 100^{m}, such that as the valve 100^{m} expands in the radial direction and foreshortens in the axial direction, the outflow end 104 will tend to move toward the inflow end 106, advantageously keeping the inflow end 106 fixed in position relative to the native annulus 24.

In some examples, a prosthetic valve 100 can optionally include only inflow vertical struts 116 without any outflow struts, which will result in a similar effect of the outflow end 104 moving towards the inflow end 106 during frame 102 foreshortening, due to the increased rigidity of the frame's inflow end portion.

It is to be understood that features of prosthetic valve 100ⁱ, equipped with longer inflow vertical struts 116, can optionally be used in combination with features and components of any of the exemplary delivery assemblies 200 disclosed herein, including delivery assemblies 200^{b}, 200^{c}, 200^{b}, 200^{d}, 200^{e}, 200^{h}, 200ⁱ, 200^{j}, and/or 200^{k}, as well as prosthetic valves 100 and/or 100^{l}. Similarly, any other features or components of any of the exemplary delivery assemblies 200 and/or prosthetic valves 100 can be used in combination with each other.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A delivery assembly comprising: a prosthetic valve comprising a frame movable between a radially compressed and a radially expanded configuration; and a delivery apparatus comprising: a handle; an outer delivery shaft extending distally from the handle; at least one elongated positioning member extending through and axially movable relative to the outer delivery shaft; and at least one sensor attached to the at least one elongated positioning member; wherein the at least one elongated positioning member is configured to position a distal end thereof radially outward to the prosthetic valve and axially distal to an outflow end of the frame.

Example 2. The delivery assembly of any example herein, particularly example 1, wherein the at least one elongated positioning member comprises a conductive material, and is configured to function as a communication device through which electric signals acquired by the corresponding sensor can be delivered.

Example 3. The delivery assembly of any example herein, particularly example 2, wherein the at least one elongated positioning member is insulated along a length thereof, and is exposed at the region of attachment of the sensor thereto.

Example 4. The delivery assembly of any example herein, particularly any one of examples 1 to 3, wherein the at least one sensor is oriented distally.

Example 5. The delivery assembly of any example herein, particularly example 4, wherein the at least one sensor is attached to a distal end of the elongated positioning member.

Example 6. The delivery assembly of any example herein, particularly any one of examples 1 to 3, wherein the at least one sensor is oriented laterally.

Example 7. The delivery assembly of any example herein, particularly example 6, wherein the at least one sensor is attached to a portion of the elongated positioning member configured for positioning distal to the outflow end.

Example 8. The delivery assembly of any example herein, particularly example 7, wherein the at least one sensor is configured to face radially away from the prosthetic valve when positioned distal to the outflow end.

Example 9. The delivery assembly of any example herein, particularly example 7, wherein the at least one sensor is configured to face radially towards the prosthetic valve when positioned distal to the outflow end.

Example 10. The delivery assembly of any example herein, particularly any one of examples 4 to 9, wherein the sensor is a force sensor.

Example 11. The delivery assembly of any example herein, particularly any one of examples 1 to 3, wherein the at least one sensor comprises at least two sensors coupled to the at least one elongated positioning member.

Example 12. The delivery assembly of any example herein, particularly example 11, wherein the at least two sensors are axially spaced from each other.

Example 13. The delivery assembly of any example herein, particularly example 12, wherein at least one of the at least two sensors is attached to a portion of the elongated positioning member configured for positioning distal to the outflow end, while another one of the at least two sensors is attached to a portion of the elongated positioning member configured to remain proximal to the outflow end.

Example 14. The delivery assembly of any example herein, particularly example 12, wherein the at least two sensors are attached to portions of the elongated positioning member configured to remain proximal to the outflow end during expansion of the prosthetic valve inside a native annulus.

Example 15. The delivery assembly of any example herein, particularly any one of examples 11 to 14, wherein the at least two sensors are pressure sensors.

Example 16. The delivery assembly of any example herein, particularly any one of examples 1 to 3, wherein the at least one sensor comprises a flow sensor.

Example 17. The delivery assembly of any example herein, particularly example 16, wherein the flow sensor is attached to a portion of the elongated positioning member configured to remain proximal to the outflow end during expansion of the prosthetic valve inside a native annulus.

Example 18. The delivery assembly of any example herein, particularly any one of examples 1 to 3, wherein the at least one sensor comprises a first pressure sensor, and wherein the delivery assembly further comprises a second pressure sensor attached to another component of the delivery assembly.

Example 19. The delivery assembly of any example herein, particularly example 18, wherein the first pressure sensor is attached to a portion of the elongated positioning member configured to remain proximal to the outflow end during expansion of the prosthetic valve inside a native annulus.

Example 20. The delivery assembly of any example herein, particularly example 19, wherein the second pressure sensor is attached to a portion of the another component of the delivery assembly configured to remain distal to the first pressure sensor during expansion of the prosthetic valve inside a native annulus.

Example 21. The delivery assembly of any example herein, particularly any one of examples 18 to 20, wherein the another component of the delivery assembly to which the second sensor is attached is axially movable with respect to the first pressure sensor.

Example 22. The delivery assembly of any example herein, particularly any one of examples 18 to 21, wherein the second pressure sensor is attached to the prosthetic valve.

Example 23. The delivery assembly of any example herein, particularly example 22, wherein the second pressure sensor is attached to a portion of the prosthetic valve which is closer to an inflow end of the frame than to the outflow end.

Example 24. The delivery assembly of any example herein, particularly any one of examples 18 to 21, wherein the second pressure sensor is attached to a nosecone shaft of the delivery apparatus.

Example 25. The delivery assembly of any example herein, particularly example 24, wherein the second pressure sensor is attached to a distal portion of the nosecone shaft.

Example 26. The delivery assembly of any example herein, particularly any one of examples 18 to 21, wherein the second pressure sensor is attached to a nosecone of the delivery apparatus.

Example 27. The delivery assembly of any example herein, particularly any one of examples 18 to 21, wherein the second pressure sensor is attached to a balloon catheter of the delivery apparatus.

Example 28. The delivery assembly of any example herein, particularly any one of examples 1 to 27, wherein the distal end of the at least one elongated positioning member is atraumatic.

Example 29. The delivery assembly of any example herein, particularly any one of examples 1 to 28, wherein the at least one elongated positioning member comprises a radiopaque material.

Example 30. The delivery assembly of any example herein, particularly any one of examples 1 to 28, wherein the at least one elongated positioning member comprises a loop, the loop defining two side segments and a bottom curved segment.

Example 31. The delivery assembly of any example herein, particularly example 30, wherein the loop is formed of a shape memory material, configured to adopt an expanded predetermined shape when not constricted inside the outer delivery shaft.

Example 32. The delivery assembly of any example herein, particularly example 30 or 31, wherein the at least one sensor is attached to the bottom curved segment.

Example 33. The delivery assembly of any example herein, particularly example 30 or 31, wherein the at least one sensor is attached to one of the side segments.

Example 34. The delivery assembly of any example herein, particularly any one of examples 30 to 33, wherein the loop comprises a radiopaque material.

Example 35. The delivery assembly of any example herein, particularly any one of examples 1 to 34, wherein the prosthetic valve further comprises a valvular structure comprising a plurality of leaflets disposed inside the frame and configured to regulate flow of blood through the prosthetic valve.

Example 36. The delivery assembly of any example herein, particularly example 35, wherein the plurality of leaflets comprises three leaflets.

Example 37. A method comprising: advancing a delivery assembly that comprises a delivery apparatus carrying a prosthetic valve in a radially compressed configuration, to a native heart valve; extending at least one elongated positioning member of the delivery apparatus through and distally to an outer delivery shaft of the delivery apparatus, until a distal end of the at least one elongated positioning member interacts with a proximal abutment surface of the native heart valve; acquiring measurement signals from at least one sensor attached to the at least one elongated positioning member; and expanding the prosthetic valve within an annulus of the native heart valve, while the distal end of the at least one elongated positioning member is radially spaced away from the prosthetic valve.

Example 38. The method of any example herein, particularly example 37, wherein the expanding the prosthetic valve within the annulus comprises advancing the prosthetic valve out of the outer delivery shaft, into the annulus.

Example 39. The method of any example herein, particularly example 38, wherein the advancing the prosthetics valve is performed simultaneously with the extending the at least one elongated positioning member.

Example 40. The method of any example herein, particularly example 38, wherein the advancing the prosthetics valve is sequential to the extending the at least one elongated positioning member.

Example 41. The method of any example herein, particularly example 38, wherein the expanding the prosthetic valve within the annulus comprises partially expanding the prosthetic valve to a diameter which is less than the diameter of the annulus, pausing valve expansion, and fully expanding the prosthetic valve against the native annulus, and wherein the method further comprises, subsequent to partially expanding the prosthetic valve and prior to fully expanding the prosthetic valve, retracting the at least one elongated positioning member from the native heart valve.

Example 42. The method of any example herein, particularly any one of examples 38 to 41, wherein the extending the at least one elongated positioning member comprises identifying a position of the proximal abutment surface of the native heart valve by monitoring the at least one elongated positioning member under fluoroscopy.

Example 43. The method of any example herein, particularly example 42, wherein the advancing the prosthetic valve comprises positioning an inflow end of the prosthetic valve at an axial position relative to the at least one elongated positioning member while monitoring both a frame of the prosthetic valve and the at least one elongated positioning member under fluoroscopy.

Example 44. The method of any example herein, particularly any one of examples 37 to 43, wherein the at least one sensor is a distally oriented force sensor configured to contact the proximal abutment surface.

Example 45. The method of any example herein, particularly example 44, wherein the acquiring the measurement signals is performed during the extension of the at least one elongated positioning member through the outer delivery shaft, and wherein interaction of the at least one elongated positioning member with the proximal abutment surface is identified by a force measured by the force sensor, indicative of contacting the proximal abutment surface.

Example 46. The method of any example herein, particularly any one of examples 37 to 43, wherein the at least one sensor is a laterally oriented force sensor positioned between the prosthetic valve and an aortic root inner wall during expansion of the prosthetic valve within the annulus.

Example 47. The method of any example herein, particularly example 46, wherein the acquiring the measurement signals is performed during the expansion of the prosthetic valve, and wherein the measurement signals are indicative of the force exerted by the prosthetic valve against the annulus.

Example 48. The method of any example herein, particularly example 46 or 47, wherein the force sensor is oriented towards the aortic root inner wall.

Example 49. The method of any example herein, particularly example 46 or 47, wherein the force sensor is oriented towards the prosthetic valve.

Example 50. The method of any example herein, particularly any one of examples 37 to 49, wherein the at least one elongated positioning member is radiopaque, and wherein the extending the at least one elongated positioning member comprises identifying, under image guidance, deformation of the elongated positioning member due to contact with the proximal abutment surface.

Example 51. A delivery assembly comprising: a prosthetic valve comprising a frame movable between a radially compressed and a radially expanded configuration; and a delivery apparatus comprising: a handle; an outer delivery shaft extending distally from the handle; a positioning balloon movable between a deflated state and an inflated state; and an inflation tube coupled to and in fluid communication with the positioning balloon, the inflation tube extending through and axially movable relative to the outer delivery shaft; wherein the inflation tube is configured to position the positioning balloon radially outward to the prosthetic valve, and such that at least a portion of the positioning balloon extends axially distally to an outflow end of the frame.

Example 52. The delivery assembly of any example herein, particularly example 51, wherein a distal end of the positioning balloon forms an atraumatic surface in the inflated state.

Example 53. The delivery assembly of any example herein, particularly example 51 or 52, wherein the inflation tube is configured to deliver inflation fluid into the positioning balloon to transition the balloon to the inflated state, and to evacuate inflation fluid from the positioning balloon when the positioning balloon transitions to the deflated state.

Example 54. The delivery assembly of any example herein, particularly any one of examples 51 to 53, wherein the positioning balloon, when positioned in an inflated state between the prosthetic valve and an aortic root inner wall surrounding the prosthetic valve, is configured to deflate in response to being squeezed between the prosthetic valve and the aortic root inner wall during expansion of the prosthetic valve.

Example 55. The delivery assembly of any example herein, particularly any one of examples 51 to 54, wherein, in the inflated state of the positioning balloon, the internal pressure of the positioning balloon is low enough to allow deformation of the positioning balloon when squeezed by the prosthetic valve during expansion of the prosthetic valve.

Example 56. The delivery assembly of any example herein, particularly any one of examples 51 to 55, wherein the balloon comprises a radiopaque material.

Example 57. The delivery assembly of any example herein, particularly any one of examples 51 to 56, wherein the prosthetic valve further comprises a valvular structure comprising a plurality of leaflets disposed inside the frame and configured to regulate flow of blood through the prosthetic valve.

Example 58. The delivery assembly of any example herein, particularly example 57, wherein the plurality of leaflets comprises three leaflets.

Example 59. A delivery assembly comprising: a prosthetic valve comprising a frame movable between a radially compressed and a radially expanded configuration; and a delivery apparatus comprising: a handle; an outer delivery shaft extending distally from the handle; and a stabilization filter configured to transition between collapsed state and a deployed state, positioned proximal to the prosthetic valve, wherein the stabilization filter comprises a plurality of pores.

Example 60. The delivery assembly of any example herein, particularly example 59, wherein the stabilization filter comprises a braided mesh.

Example 61. The delivery assembly of any example herein, particularly example 59 or 60, wherein the stabilization filter is made of a shape-memory material, shape-set to self-expand to the deployed state when not restricted by an outer shaft.

Example 62. The delivery assembly of any example herein, particularly example 61, wherein the shape-memory material is Nitinol.

Example 63. The delivery assembly of any example herein, particularly any one of examples 59 to 62, wherein the maximal size of the pores is between 40 and 300 microns.

Example 64. The delivery assembly of any example herein, particularly any one of examples 59 to 62, wherein the average size of the pores is between 40 and 300 microns.

Example 65. The delivery assembly of any example herein, particularly any one of examples 59 to 64, wherein the stabilization filter is attached to a shaft of the delivery apparatus, which is axially movable through the outer delivery shaft.

Example 66. The delivery assembly of any example herein, particularly example 65, wherein the shaft to which the stabilization filter is attached is a balloon catheter, and wherein the delivery apparatus further comprises a balloon attached to the balloon catheter.

Example 67. The delivery assembly of any example herein, particularly example 66, wherein the stabilization filter is attached to a portion of the balloon catheter which is proximal to the balloon.

Example 68. The delivery assembly of any example herein, particularly any one of examples 65 to 67, wherein the stabilization filter does not include any openings between an outermost diameter thereof, in the deployed state, and the shaft it is attached to, which are greater in size than the pores.

Example 69. The delivery assembly of any example herein, particularly any one of examples 65 to 68, wherein the stabilization filter comprises a radiopaque material.

Example 70. The delivery assembly of any example herein, particularly any one of examples 59 to 69, wherein the prosthetic valve further comprises a valvular structure comprising a plurality of leaflets disposed inside the frame and configured to regulate flow of blood through the prosthetic valve.

Example 71. The delivery assembly of any example herein, particularly example 70, wherein the plurality of leaflets comprises three leaflets.

Example 72. A delivery assembly comprising: a prosthetic valve comprising: a frame movable between a radially compressed and a radially expanded configuration, the frame extending between an inflow end and an outflow end; and one or more positioning struts configured to transition between a compacted state and a deployed state; and a delivery apparatus comprising: a handle; an inner capsule configured to retain the prosthetic valve in the radially compressed configuration therein; and an outer capsule having an inner diameter greater than an outer diameter of the inner capsule; wherein the one or more positioning struts are configured to extend radially away from the frame in the deployed state; and wherein the prosthetic valve, the inner capsule, and the outer capsule, are axially movable relative to each other.

Example 73. The delivery assembly of any example herein, particularly example 72, wherein the one or more positioning struts are coupled, at fixed ends thereof, to junctions of the frame which are closer to the inflow end than to the outflow end.

Example 74. The delivery assembly of any example herein, particularly example 72, wherein the one or more positioning struts are coupled, at fixed ends thereof, to inflow apices of the frame.

Example 75. The delivery assembly of any example herein, particularly any one of examples 72 to 74, wherein the one or more positioning struts terminate at free ends which are atraumatic.

Example 76. The delivery assembly of any example herein, particularly example 75, wherein the free ends are configured to be positioned proximal to the inflow end in the deployed state.

Example 77. The delivery assembly of any example herein, particularly any one of examples 72 to 76, wherein the one or more positioning struts are formed from a shape-memory material.

Example 78. The delivery assembly of any example herein, particularly example 77, wherein the shape-memory material is Nitinol.

Example 79. The delivery assembly of any example herein, particularly any one of examples 72 to 78, wherein the one or more positioning struts are integrally formed with the frame.

Example 80. The delivery assembly of any example herein, particularly any one of examples 72 to 79, wherein the inner capsule comprises one or more slots extending proximally from a distal end of the inner capsule.

Example 81. The delivery assembly of any example herein, particularly example 80, wherein the number of the slots 78 matches the number of the positioning struts.

Example 82. The delivery assembly of any example herein, particularly example 80 or 81, wherein the one or more slots are circumferentially aligned with the one or more positioning struts.

Example 83. The delivery assembly of any example herein, particularly any one of examples 80 to 82, wherein the one or more slots are sized to allow the positioning struts to extends therethrough.

Example 84. The delivery assembly of any example herein, particularly any one of examples 72 to 83, wherein the one or more positioning struts are pre-shaped to assume a curved configuration in their deployed state.

Example 85. The delivery assembly of any example herein, particularly example 84, wherein the curved configuration is C-shaped or U-shaped.

Example 86. The delivery assembly of any example herein, particularly any one of examples 72 to 85, wherein the one or more positioning struts comprises a radiopaque material.

Example 87. The delivery assembly of any example herein, particularly any one of examples 72 to 86, wherein the prosthetic valve further comprises a valvular structure comprising a plurality of leaflets disposed inside the frame and configured to regulate flow of blood through the prosthetic valve.

Example 88. The delivery assembly of any example herein, particularly example 87, wherein the plurality of leaflets comprises three leaflets.

Example 89. A delivery assembly comprising: a prosthetic valve comprising a frame movable between a radially compressed and a radially expanded configuration, the frame extending between an inflow end and an outflow end; and a delivery apparatus comprising: a handle; a nosecone shaft extending distally from the handle, the nosecone shaft defining a nosecone shaft lumen and comprising one or more side opening formed at a distal portion of the nosecone shaft; a nosecone attached to the distal portion of the nosecone shaft; and one or more positioning arms extending through the nosecone shaft lumen, the one or more positioning arms configured to transition between a compacted state and a deployed state; wherein the one or more positioning arms are axially movable relative to the nosecone shaft and the nosecone, and are configured to assume the compacted state when fully retained inside the nosecone shaft lumen, and to assume a deployed state when at least a portion thereof extends through the one or more side openings; and wherein the one or more positioning arms are configured to extend radially away from the nosecone shaft in the deployed state.

Example 90. The delivery assembly of any example herein, particularly example 89, wherein the one or more positioning arms terminate at free ends which are atraumatic.

Example 91. The delivery assembly of any example herein, particularly example 90, wherein the free ends are configured to be positioned proximal to the inflow end of the frame in the deployed state.

Example 92. The delivery assembly of any example herein, particularly example 90 or 91, wherein the free ends are configured to be to be offset radially away from the frame in the deployed state.

Example 93. The delivery assembly of any example herein, particularly any one of examples 89 to 92, wherein the nosecone shaft and the prosthetic valve are axially movable relative to each other, such that the one or more side openings are configured to be positioned distally to the inflow end of the frame.

Example 94. The delivery assembly of any example herein, particularly any one of examples 89 to 93, wherein the one or more positioning arms are formed from a shape-memory material.

Example 95. The delivery assembly of any example herein, particularly example 94, wherein the shape-memory material is Nitinol.

Example 96. The delivery assembly of any example herein, particularly any one of examples 89 to 95, wherein the one or more positioning arms are pre-shaped to assume a curved configuration in their deployed state.

Example 97. The delivery assembly of any example herein, particularly example 96, wherein the curved configuration is C-shaped or U-shaped.

Example 98. The delivery assembly of any example herein, particularly any one of examples 89 to 97, wherein the number of the side openings matches the number of the positioning arms.

Example 99. The delivery assembly of any example herein, particularly any one of examples 89 to 98, wherein the one or more side openings are circumferentially aligned with the one or more positioning arms.

Example 100. The delivery assembly of any example herein, particularly any one of examples 89 to 99, wherein the one or more side openings are sized to allow the positioning arms to extends therethrough.

Example 101. The delivery assembly of any example herein, particularly any one of examples 89 to 100, wherein the one or more positioning arms comprises a radiopaque material.

Example 102. The delivery assembly of any example herein, particularly any one of examples 89 to 101, wherein the prosthetic valve further comprises a valvular structure comprising a plurality of leaflets disposed inside the frame and configured to regulate flow of blood through the prosthetic valve.

Example 103. The delivery assembly of any example herein, particularly example 102, wherein the plurality of leaflets comprises three leaflets.

Example 104. The delivery assembly of any example herein, particularly example 102 or 103, wherein the nosecone shaft extends through the prosthetic valve, between the plurality of leaflets.

Example 105. A delivery assembly comprising: a prosthetic valve comprising a frame movable between a radially compressed and a radially expanded configuration, the frame extending between an inflow end and an outflow end; and a delivery apparatus comprising: a handle; a nosecone shaft extending distally from the handle, the nosecone shaft defining a nosecone shaft lumen and comprising one or more side opening formed at a distal portion of the nosecone shaft; a nosecone attached at a nosecone proximal end thereof to the distal portion of the nosecone shaft; one or more positioning arms attached to the nosecone at fixed ends thereof and extending proximally therefrom to free ends of the one or more positioning arms, wherein the one or more positioning arms configured to transition between a compacted state and a deployed state; and one or more tensioning members attached to the free ends of the one or more positioning arms, and extending proximally therefrom; wherein the one or more positioning arms are configured to assume the compacted state when the one or more tensioning members attached thereto are tensioned, and to assume the deployed state when tension is released from the one or more tensioning members; and wherein the one or more positioning arms are configured to extend radially away from the nosecone and the nosecone shaft in the deployed state.

Example 106. The delivery assembly of any example herein, particularly example 105, wherein the free ends of the one or more positioning arms are atraumatic.

Example 107. The delivery assembly of any example herein, particularly example 105 or 106, wherein the free ends are configured to be positioned proximal to the inflow end of the frame in the deployed state.

Example 108. The delivery assembly of any example herein, particularly any one of examples 105 to 107, wherein the free ends are configured to be to be offset radially away from the frame in the deployed state.

Example 109. The delivery assembly of any example herein, particularly any one of examples 105 to 108, wherein the nosecone and the prosthetic valve are axially movable relative to each other, such that the one or more free ends are configured to be positioned distally to the inflow end of the frame.

Example 110. The delivery assembly of any example herein, particularly any one of examples 105 to 109, wherein the one or more positioning arms are formed from a shape-memory material.

Example 111. The delivery assembly of any example herein, particularly example 110, wherein the shape-memory material is Nitinol.

Example 112. The delivery assembly of any example herein, particularly any one of examples 105 to 111, wherein the one or more positioning arms are pre-shaped to assume a curved configuration in their deployed state.

Example 113. The delivery assembly of any example herein, particularly any one of examples 105 to 112, wherein the number of the tensioning members matches the number of the positioning arms.

Example 114. The delivery assembly of any example herein, particularly any one of examples 105 to 113, wherein the one or more tensioning members extend from the free ends of the positioning arms, through the one or more side openings, into the nosecone shaft lumen.

Example 115. The delivery assembly of any example herein, particularly example 114, wherein the one or more tensioning members extend inside the nosecone shaft lumen, from the side openings to the handle.

Example 116. The delivery assembly of any example herein, particularly any one of examples 105 to 115, wherein the one or more side openings are circumferentially aligned with the one or more free ends of the positioning arms.

Example 117. The delivery assembly of any example herein, particularly any one of examples 105 to 116, wherein the one or more side openings are sized to allow the tensioning members to extends therethrough.

Example 118. The delivery assembly of any example herein, particularly any one of examples 105 to 117, wherein the one or more positioning arms comprises a radiopaque material.

Example 119. The delivery assembly of any example herein, particularly any one of examples 105 to 118, wherein the one or more positioning arms are attached, at their fixed ends, to the nosecone proximal end.

Example 120. The delivery assembly of any example herein, particularly any one of examples 105 to 119, wherein the one or more tensioning members comprise at least one of: a wire, a string, a suture, and/or a cable.

Example 121. The delivery assembly of any example herein, particularly any one of examples 105 to 120, wherein the one or more positioning arms are parallel to the nosecone shaft in the compacted state.

Example 122. The delivery assembly of any example herein, particularly any one of examples 105 to 121, wherein the prosthetic valve further comprises a valvular structure comprising a plurality of leaflets disposed inside the frame and configured to regulate flow of blood through the prosthetic valve.

Example 123. The delivery assembly of any example herein, particularly example 122, wherein the plurality of leaflets comprises three leaflets.

Example 124. The delivery assembly of any example herein, particularly example 122 or 123, wherein the nosecone shaft extends through the prosthetic valve, between the plurality of leaflets.

Example 125. A delivery assembly comprising: a prosthetic valve comprising: a frame movable between a radially compressed and a radially expanded configuration; and an outer skirt disposed around the frame, the outer skirt comprising a circumferential mesh configured to transition between a compacted state and an expanded free state; and a delivery apparatus comprising a capsule; wherein the circumferential mesh is configured to assume the compacted state when the prosthetic valve is retained inside the capsule, and to assume the expanded free state when the prosthetic valve is deployed out of the capsule.

Example 126. The delivery assembly of any example herein, particularly example 125, wherein the outer skirt further comprises a base layer, wherein the base layer is attached to the frame, and wherein the circumferential mesh is attached to the base layer.

Example 127. The delivery assembly of any example herein, particularly example 125 or 126, wherein the circumferential mesh extends around an inflow portion of the frame.

Example 128. The delivery assembly of any example herein, particularly any one of examples 125 to 127, wherein the circumferential mesh comprises a flexible braided material.

Example 129. The delivery assembly of any example herein, particularly any one of examples 125 to 128, wherein the circumferential mesh is made of a shape-memory material, shape-set to self-expand to the expanded free state when not restricted by the capsule.

Example 130. The delivery assembly of any example herein, particularly example 129, wherein the shape-memory material is Nitinol.

Example 131. The delivery assembly of any example herein, particularly any one of examples 125 to 130, wherein a radial distance defined by the circumferential mesh between the frame and an outermost edge of the circumferential mesh, in the expanded free state, is greater than a radius defined by the frame in the expanded configuration of the frame.

Example 132. The delivery assembly of any example herein, particularly example 131, wherein the radial distance of the circumferential mesh in its expanded free state is at least as great as the diameter of the frame in the expanded configuration of the frame.

Example 133. The delivery assembly of any example herein, particularly any one of examples 125 to 132, wherein the circumferential mesh comprises a radiopaque material.

Example 134. The delivery assembly of any example herein, particularly any one of examples 125 to 133, wherein the prosthetic valve further comprises a valvular structure comprising a plurality of leaflets disposed inside the frame and configured to regulate flow of blood through the prosthetic valve.

Example 135. The delivery assembly of any example herein, particularly example 134, wherein the plurality of leaflets comprises three leaflets.

Example 136. A prosthetic valve comprising: a frame movable between a radially compressed and a radially expanded configuration, the frame extending between an inflow end and an outflow end, and comprising a plurality of intersecting struts, the plurality of struts comprising: a plurality of angled struts; a plurality of vertical struts comprising: a plurality of inflow vertical struts defined between cells of the frame extending from the inflow end; and a plurality of outflow vertical struts defined between cells of the frame extending from the outflow end; wherein the inflow vertical struts define an inflow strut length which is greater than an outflow struts length defined by the outflow vertical struts.

Example 137. The prosthetic valve of any example herein, particularly example 136, further comprises a valvular structure comprising a plurality of leaflets disposed inside the frame and configured to regulate flow of blood through the prosthetic valve.

Example 138. The prosthetic valve of any example herein, particularly example 137, wherein the plurality of leaflets comprises three leaflets.

Example 139. The prosthetic valve of any example herein, particularly example 137 or 138, further comprising a plurality of commissures formed between adjacent leaflets, wherein the plurality of commissures are coupled to at least some of the plurality of outflow vertical struts.

Example 140. The prosthetic valve of any example herein, particularly any one of examples 137 to 139, wherein the cells extending from the outflow end and the cells extending from the inflow end define hexagonal openings.

Example 141. The prosthetic valve of any example herein, particularly example 140, wherein the hexagonal openings of the cells extending from the inflow end are larger than the hexagonal openings of the cells extending from the outflow end.

Example 142. The prosthetic valve of any example herein, particularly any one of examples 137 to 141, further comprising an outer skirt disposed around an outer surface of the frame.

Example 143. The prosthetic valve of any example herein, particularly any one of examples 137 to 142, further comprising an inner skirt disposed around an inner surface of the frame.

Example 144. The prosthetic valve of any example herein, particularly any one of examples 137 to 143, wherein the frame comprises at least one additional row of diamond-shaped cells, disposed between the cells extending from the inflow end and the cells extending from the outflow end.

Example 145. The prosthetic valve of any example herein, particularly any one of examples 137 to 144, wherein the frame comprises a cobalt-chromium alloy.

Example 146. The prosthetic valve of any example herein, particularly any one of examples 137 to 145, wherein the frame further comprises a plurality of inflow apices at the inflow end and a plurality of outflow apices at the outflow end, wherein each inflow apex is positioned angularly between two of the plurality of inflow vertical struts, and wherein each outflow apex is positioned angularly between two of the plurality of outflow vertical struts.

Example 147. The prosthetic valve of any example herein, particularly any one of examples 137 to 146, wherein the angled struts are angled relative to a central longitudinal axis of the frame, and wherein the vertical struts are parallel to the central longitudinal axis.

Example 148. The delivery assembly of any example herein, particularly any one of examples 1-36 or 39-135, wherein the frame comprises a plurality of struts, the plurality of struts comprising: a plurality of angled struts; a plurality of vertical struts comprising: a plurality of inflow vertical struts defined between cells of the frame extending from an inflow end of the frame; and a plurality of outflow vertical struts defined between cells of the frame extending from an outflow end of the frame; wherein the inflow vertical struts define an inflow strut length which is greater than an outflow struts length defined by the outflow vertical struts.

Example 149. The delivery assembly of any example herein, particularly example 148, wherein the cells extending from the outflow end and the cells extending from the inflow end define hexagonal openings.

Example 150. The delivery assembly of any example herein, particularly example 149, wherein the hexagonal openings of the cells extending from the inflow end are larger than the hexagonal openings of the cells extending from the outflow end.

Example 151. A method comprising: advancing a delivery assembly that comprises a delivery apparatus carrying a prosthetic valve in a radially compressed configuration, to a native heart valve; contacting an abutment surface of the native heart valve by at least one positioning member of the delivery apparatus; identifying axial position of an annulus of the native heart valve by monitoring the at least one positioning member under fluoroscopy; positioning an inflow end of the prosthetic valve at an axial position relative to the at least one positioning member while monitoring both a frame of the prosthetic valve and the at least one positioning member under fluoroscopy; and expanding the prosthetic valve within the annulus.

Example 152. The method of any example herein, particularly example 151, wherein the prosthetic valve is axially movable relative to the at least one positioning member.

Example 153. The method of any example herein, particularly example 152, wherein the positioning the inflow end of the prosthetic valve comprises axially moving the prosthetic valve relative to the at least one positioning member, while maintaining position of the at least one positioning member in contact with the abutment surface.

Example 154. The method of any example herein, particularly any one of examples 151 to 153, wherein the abutment surface is a proximal abutment surface of the native heart valve.

Example 155. The method of any example herein, particularly any one of examples 151 to 153, wherein the abutment surface is a distal abutment surface of the native heart valve.

Example 156. The method of any example herein, particularly any one of examples 151 to 155, wherein the contacting the abutment surface comprises using tactile feedback from the at least one positioning member to confirm contact of the at least one positioning member with the abutment surface.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

What is claimed is:

1. A delivery assembly comprising:
a prosthetic valve comprising:
a frame movable between a radially compressed and a radially expanded configuration, the frame extending between an inflow end and an outflow end; and
one or more positioning struts configured to transition between a compacted state and a deployed state; and
a delivery apparatus comprising:
a handle;
an inner capsule configured to retain the prosthetic valve in the radially compressed configuration therein; and
an outer capsule having an inner diameter greater than an outer diameter of the inner capsule;
wherein the one or more positioning struts are configured to extend radially away from the frame in the deployed state; and
wherein the prosthetic valve, the inner capsule, and the outer capsule, are axially movable relative to each other.

2. The delivery assembly of claim 1, wherein the one or more positioning struts are coupled, at fixed ends thereof, to one or more junctions of the frame which are closer to the inflow end than to the outflow end.

3. The delivery assembly of claim 1, wherein the one or more positioning struts are coupled, at fixed ends thereof, to one or more inflow apices of the frame.

4. The delivery assembly of claim 1, wherein the one or more positioning struts terminate at free ends which are atraumatic.

5. The delivery assembly of claim 4, wherein the free ends are positioned proximal to the inflow end in the deployed state.

6. The delivery assembly of claim 1, wherein the one or more positioning struts are formed from a shape-memory material.

7. The delivery assembly of claim 6, wherein the shape-memory material comprises Nitinol.

8. The delivery assembly of claim 1, wherein the one or more positioning struts are integrally formed with the frame.

9. The delivery assembly of claim 1, wherein the inner capsule comprises one or more slots extending proximally from a distal end of the inner capsule.

10. The delivery assembly of claim 9, wherein the number of the slots matches the number of the positioning struts.

11. The delivery assembly of claim 9, wherein the one or more slots are circumferentially aligned with the one or more positioning struts.

12. The delivery assembly of claim 9, wherein the one or more slots are sized to allow the positioning struts to extends therethrough.

13. The delivery assembly of claim 1, wherein the one or more positioning struts are pre-shaped to assume a curved configuration in their deployed state.

14. The delivery assembly of claim 13, wherein the curved configuration is C-shaped or U-shaped.

15. The delivery assembly of claim 1, wherein the one or more positioning struts comprises a radiopaque material.

16. The delivery assembly of claim 1, wherein the prosthetic valve further comprises a valvular structure comprising a plurality of leaflets disposed inside the frame and configured to regulate flow of blood through the prosthetic valve.

17. The delivery assembly of claim 16, wherein the plurality of leaflets comprises three leaflets.

18. The delivery assembly of claim 1, wherein axial movement of the outer capsule relative to the inner capsule is configured to allow the one or more positioning struts to transition to the deployed state while the frame is maintained in the radially compressed configuration inside the inner capsule.

19. The delivery assembly of claim 1, wherein the one or more positioning struts are configured to extend along a space defined between the inner capsule and the outer capsule in a delivery configuration in which the frame is in the radially compressed configuration and the one or more positioning struts are in the compacted state.

20. The delivery assembly of claim 1, wherein each of the inner capsule and the outer capsule is independently axially movable relative to the prosthetic valve.
